(19) United States
(12) Patent Application Publication
(10) Pub. No.: US 2026/0102340 A1
(43) Pub. Date: Apr. 16, 2026

(54) Therapeutic Formulation

(71) Applicant: 1000739373 Ontario Ltd., Sudbury (CA)

(72) Inventors: David Joseph Rodney, Sudbury (CA); Patrick Lehoux, Sudbury (CA); Mitchell Thompson, Sudbury (CA); Angelo Psellas, Komoka (CA)

(21) Appl. No.: 19/114,957

(22) PCT Filed: Sep. 26, 2023
(86) PCT No.: PCT/IB2023/059545

§ 371 Date: Mar. 25, 2025

Related U.S. Application Data
(60) Provisional application No. 63/410,007, filed on Sep. 26, 2022.

(51) Int. Cl. A61K 9/00; A61K 31/00; A61K 31/4045; A61K 31/465; A61K 31/522; A61K 47/06; A61K 47/10; A61K 47/22

(52) U.S. Cl. CPC A61K 9/008; A61K 9/006; A61K 31/4045; A61K 31/465; A61K 31/522; A61K 31/658; A61K 47/06; A61K 47/10; A61K 47/22

(57) ABSTRACT

Ingestion of therapeutic agents are characterized by low bioavailability, a lengthy onset of action, and inconsistent absorption. A therapeutic formulation for pulmonary or sublingual administration of an active agent is disclosed. The therapeutic formulation includes an active agent that can be absorbed through the pulmonary membrane or sublingual tissues for rapid absorption and high bioavailability. In particular examples, the active agent includes nicotine or melatonin. The therapeutic formulation further includes a co-solvent and a propellant.

### CROSS-REFERENCE TO RELATED APPLICATIONS

This application is the United States national phase of International Patent Application No. PCT/IB2023/059545 filed Sep. 26, 2023, and claims priority to U.S. Provisional Patent Application No. 63/410,007 filed Sep. 26, 2022, the disclosures of which are hereby incorporated by reference in their entireties.

### BACKGROUND OF THE INVENTION

### Field of the Invention

The present specification is directed to a therapeutic formulation for use in an inhalation device or sublingual spray device.

### Description of Related Art

Most therapeutic agents are administered orally, typically as a tablet. However, absorption of orally administered drugs is largely inconsistent. Absorption can be affected by other drugs, food, and drink consumed within hours of the drug. Drug oral bioavailability is also very low, with only a fraction of the dose reaching the therapeutic site of action. Furthermore, the onset of action can range between 20 minutes to over an hour, depending on the drug. This delay can make it difficult to assess the drug's effects and determine whether an additional dose is needed.

### SUMMARY OF THE INVENTION

An aspect of the specification provides a therapeutic formulation for pulmonary or sublingual delivery of an active agent. The therapeutic formulation includes a therapeutically effective amount of the active agent, a co-solvent, and a propellant.

In some examples, the active agent includes nicotine. The formulation may include 0.225 to 3% nicotine by weight. In particular examples, the formulation includes 0.77±0.08% nicotine by weight. In some examples, the propellant includes hydrofluoroalkane 134a. In further examples, the propellant includes a mixture of hydrofluoroalkane 152a and hydrofluoroalkane 134a. In further examples, the propellant includes hydrofluoroalkane 152a. The propellant may comprise between 80% and 98% of the formulation by weight. In particular examples, the propellant comprises 95.92±9% of the formulation by weight. In some examples, the co-solvent includes ethanol. The co-solvent may comprise between 0% and 15% of the formulation by weight. In some examples, the therapeutic formulation further includes an anti-nucleating agent. The anti-nucleating agent may include propylene glycol. In further examples, the formulation includes a flavouring agent. The flavouring agent may comprise between 0 to 3% of the formulation by weight. The flavouring agent may include menthol. The menthol may comprise 0.77±0.08% by weight of the formulation.

Another aspect of the specification provides a therapeutic formulation for sublingual or pulmonary delivery of an active agent, including 95.92±9% by weight hydrofluoroalkane 152a; 1.70±0.2% by weight ethanol; 0.41±0.04% by weight water; 0.74±0.07% by weight nicotine; 0.77±0.08% by weight menthol; and 0.46±0.05% by weight of a flavouring agent.

A further aspect of the specification provides a therapeutic formulation for pulmonary or sublingual delivery of an active agent. The therapeutic formulation includes a therapeutically effective amount of melatonin, a co-solvent, and a propellant.

The formulation may include 0.01 to 1.46% (w/w) melatonin by weight of the formulation. In some examples, the propellant includes hydrofluoroalkane 134a. In further examples, the propellant includes a mixture of hydrofluoroalkane 152a and hydrofluoroalkane 134a. In further examples, the propellant includes hydrofluoroalkane 152a. The propellant may comprise between 80% and 98% of the formulation by weight. In particular examples, the propellant comprises 95.92±9% of the formulation by weight. In some examples, the co-solvent includes ethanol. The co-solvent may comprise between 0% and 15% of the formulation by weight. In some examples, the therapeutic formulation further includes an anti-nucleating agent. The anti-nucleating agent may include propylene glycol. In further examples, the formulation includes a flavouring agent. The flavouring agent may comprises between 0 to 3% of the formulation by weight. The flavouring agent may include menthol. The menthol may comprise 0.77±0.08% by weight of the formulation.

A further aspect of the specification provides a therapeutic formulation for sublingual or pulmonary delivery of an active agent, the therapeutic formulation including 6±0.6% ethanol by weight of the formulation, 0.002±0.0002% melatonin by weight of the formulation, and hydrofluoroalkane 152a.

A further aspect of the specification provides a therapeutic formulation for pulmonary delivery of an active agent. The therapeutic formulation includes 94.69±0.5% hydrofluoroalkane 152a by weight of the formulation, 5±1% ethanol by weight of the formulation, 0.1±0.1% glycerol by weight of the formulation, 0.04±0.05% saccharin by weight of the formulation, and 0.224%±0.3 nicotine by weight of the formulation.

A further aspect of the specification provides a therapeutic formulation for pulmonary delivery of an active agent. The therapeutic formulation includes 81.5±3% hydrofluoroalkane 152a by weight of the formulation, 15±1% ethanol by weight of the formulation, 2.2±1% water by weight of the formulation, and 1.3±1% caffeine by weight of the formulation.

A further aspect of the specification provides a therapeutic formulation for pulmonary delivery of an active agent. The therapeutic formulation includes 91.5±1% hydrofluoroalkane 152a by weight of the formulation, 8±3.0% ethanol by weight of the formulation, and 0.449±0.5% melatonin by weight of the formulation.

A further aspect of the specification provides a therapeutic formulation for sublingual delivery of an active agent. The therapeutic formulation includes 89±5% hydrofluoroalkane 152a by weight of the formulation, 2.34±2.5% ethanol by weight of the formulation, 7.3±3% cannabidiol by weight of the formulation, 0.06±0.05% saccharin by weight of the formulation, and 0.84%±0.3 flavoring agents by weight of the formulation.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a perspective view of a metered dose inhaler according to one aspect of the invention.

FIG. 2 is a longitudinal cross-section of the inhaler of FIG. 1 including a stem block.

FIG. 3 is an exploded view of the inhaler of FIG. 1.

FIG. 4 is a front perspective view of the stem block of FIG. 2.

FIG. 5 is a top view of the stem block of FIG. 2.

FIG. 6 is a bottom view of the stem block of FIG. 2.

FIG. 7 is a rear perspective view of the stem block of FIG. 2.

FIG. 8 is a side view of the stem block of FIG. 2.

FIG. 9 is a cross-section of a stem block according to the prior art.

FIG. 10 is a cross-section of the stem block of FIG. 2.

FIG. 11A is a cross-section of another stem block.

FIG. 11B is a cross-section of another stem block

FIG. 12A is a cross-section of another stem block

FIG. 12B is a cross-section of another stem block

FIG. 13A is a cross-section of another stem block

FIG. 13B is a cross-section of another stem block

FIG. 14A is a perspective view of a valve assembly including the stem block of FIG. 2.

FIG. 14B is a perspective view of a housing for the valve assembly of FIG. 11A including a mouthpiece.

FIG. 14C is a perspective view of the mouthpiece of FIG. 11B.

FIG. 14D is another perspective view of the housing of FIG. 11B.

FIG. 14E is another perspective view of the valve assembly of FIG. 11A.

FIG. 15 is a graph showing the relationship between melatonin and ethanol content according to one example.

FIG. 16 is a graph showing the relationship between melatonin and ethanol content according to another example.

FIG. 17 is a graph showing the relationship between melatonin and ethanol content according to another example.

FIG. 18 is a graph showing the relationship between melatonin and ethanol content according to another example.

FIG. 19A is a perspective view of a first metering valve with a dip tube according to the examples.

FIG. 19B is a perspective view of a second metering valve without a dip tube according to the examples.

FIG. 20A is a cross-sectional view of a prior art MDI at rest.

FIG. 20B is a cross-sectional view of the prior art MDI of FIG. 20A during use.

FIG. 20C is a cross-sectional view of the prior art MDI of FIG. 20A during use.

FIG. 20D is a cross-sectional view of the prior art MDI of FIG. 20A during use.

FIG. 21 is a graph showing the relationship between cumulative undersize and upper aerodynamic diameter according to one example.

FIG. 22 is a graph showing the relationship between cumulative undersize and upper aerodynamic diameter according to another example.

FIG. 23 is a graph showing the relationship between cumulative drug mass and upper aerodynamic diameter according to another example.

FIG. 24 is a graph showing the relationship between cumulative drug mass and upper aerodynamic diameter according to another example.

FIG. 25 is a graph showing the relationship between cumulative drug mass and upper aerodynamic diameter according to another example.

FIG. 26 is a graph showing the relationship between cumulative drug mass and upper aerodynamic diameter according to another example.

FIG. 27 is a graph showing the relationship between cumulative drug mass and upper aerodynamic diameter according to another example.

FIG. 28 is a graph showing the relationship between cumulative drug mass and upper aerodynamic diameter according to another example.

FIG. 29 is a graph showing the relationship between cumulative drug mass and upper aerodynamic diameter according to another example.

FIG. 30 is a graph showing the relationship between cumulative drug mass and upper aerodynamic diameter according to another example.

FIG. 31 is a graph showing the relationship between cumulative drug mass and upper aerodynamic diameter according to another example.

FIG. 32 is a graph showing the relationship between cumulative mass undersize and upper aerodynamic particle diameter according to another example.

FIG. 33 is a graph showing the relationship between cumulative mass undersize and upper aerodynamic particle diameter according to another example.

FIG. 34 is a graph showing the relationship between cumulative mass undersize and upper aerodynamic dimeter according to another example.

FIG. 35 is a graph showing the relationship between cumulative undersize and upper aerodynamic diameter according to another example.

### DESCRIPTION OF THE INVENTION

TABLE OF ABBREVIATIONS

| | |
|---|---|
| BDP | budesonide dipropionate |
| CBD | cannabidiol |
| DUSA | dose uniformity sampling apparatus |
| FPD | Fine Particle Dose |
| FPF | Fine Particle Fraction |
| GSD | geometric standard deviation |
| HFA | hydrofluoroalkane |
| HPLC | high pressure liquid chromatography |
| MDI | metered dose inhaler |
| MMAD | mass median aerodynamic diameter |
| NGI | Next Generation Impactor |
| RH | Relative Humidity |
| SD | Standard Deviation |
| USP | United States Pharmacopeia |
| % (w/w) | percent composition, measured by weight |

### Definitions

“About” herein refers to a range of +/−20% of the numerical value that follows. In one embodiment, the term “about” refers to a range of +/−10% of the numerical value that follows. In one embodiment, the term “about” refers to a range of +/−5% of the numerical value that follows.

“Metered dose inhaler” is used interchangeably with “inhaler” herein to describe a device that delivers a measured amount of a pressurized therapeutic formulation for inhalation by a subject.

### Therapeutic Formulation

The present specification provides a therapeutic formulation for sublingual or pulmonary delivery of an active agent. The therapeutic formulation provides rapid drug effects, allowing users to administer the drug at the time of need. This will improve patient compliance rates and make it easier for occasional users to enjoy the benefits of melatonin supplements. Furthermore, the rapid effects of the composition will allow users to quickly determine if a dosage was insufficient and administer an additional amount without significant delays. Users will be able to start with a lower dose and increase the dose only as required, thus reducing the incidence of overdosing. Furthermore, bioavailability for inhaled drugs is 10-200 times greater than nasal and gastrointestinal values due to its enormous surface area, and very thin diffusion layer.

The therapeutic formulation includes a therapeutically effective amount of an active agent. In particular examples described herein, the active agent is nicotine or melatonin, however the active agent is not particularly limited. The active agent may include, but is not limited to melatonin, caffeine, nicotine, taurine, Nicotinamide mononucleotide, nicotinamide riboside, ashwagandha, a cannabinoid (e.g., cannabidiol, tetrahydrocannabidiol, cannabinol, cannabigerol, tetrahydrocannabinolic acid), a compound extracted from green tea (including but not limited to epigallocatechin gallate), NAC (N-Acetylcysteine), theophylline, cetirizine, ginseng, tryptamine drugs (including but not limited to psilocybin and lysergic acid diethylamide), phenethylamine drugs (including but not limited to 3,4-methylenedioxy-methamphetamine and methamphetamine), pseudoephedrine, astragalus, a vitamin (including but not limited to vitamin C, vitamin E, vitamin B3, vitamin B6, vitamin B12, and B vitamin complex), an extract of ginger, an extract of black cohosh (Actaea racemosa), an electrolyte, sildenafil, colloidal silver, strontium, resveratrol, ghrelin, piracetam, aniracetam, oxiracetam, phenylpiracetam, noopept, alpha-glycerophosphorylcholine, cytidine diphosphate-choline (Citicoline), bacopa monnieri, Rhodiola rosea, L-theanine, huperzine A, a compound extract from lion's mane mushroom (Hericium erinaceus), an extract of Ginkgo biloba, DMT (Dimethyltryptamine), or combinations thereof.

In embodiments where the active agent comprises melatonin, melatonin may comprise about 0.01 to 1.46% (w/w) of the therapeutic formulation. The concentration of melatonin may be selected based on the predetermined dose delivered by the inhaler or spray device and the desired dose of melatonin, the desired dose being between about 100 μg and about 20 mg. Metered dose inhalers typically deliver a predetermined dose that measures between 25 and 100 μL.

The effective dosage for melatonin may vary considerably, depending on a number of factors including the individual's age, body weight, health conditions, and metabolism. Even regular users of melatonin find it difficult to guess the appropriate dose. Unfortunately, dosage errors are difficult to correct because the drug effects typically appear 30 to 60 minutes after consumption. Overdosing is therefore prevalent among users. These problems can be addressed by delivering the therapeutic formulation comprising melatonin via inhalation. Melatonin administered through the lungs is absorbed significantly faster than ingested melatonin. Pulmonary drug delivery can thus produce physiological effects within seconds or minutes of administration, allowing a user to assess and administer additional amounts if needed, without delaying their bedtime.

In embodiments where the active agent comprises nicotine, nicotine may comprise about 0.225 to about 3.0% (w/w) of the therapeutic formulation. The concentration of nicotine may be selected based on the predetermined dose delivered by the inhaler or spray device and the desired dose of nicotine, the desired dose being between about 5 mg and about 30 mg. Metered dose inhalers deliver a predetermined dose that typically measures between 25 and 100 μL.

In embodiments where the active agent comprises caffeine, caffeine may comprise about 0.1 to about 1% (w/w) of the therapeutic formulation. The concentration of caffeine may be selected based on the predetermined dose delivered by the inhaler or spray device and the desired dose of caffeine, the desired dose being between 10 mg and 100 mg. Metered dose inhalers typically deliver a predetermined dose that measures between 25 and 100 μL.

The effects of caffeine may be noticeable as early as 15 minutes after oral consumption, but the blood levels of caffeine continue to increase for up to an hour after consumption. In contrast, inhalation of the therapeutic formulation comprising caffeine provides near immediate effects. Therefore, the therapeutic formulation allows the user to better control and maintain their blood levels of caffeine.

In embodiments where the active agent comprises beclometasone dipropionate, beclometasone dipropionate may comprise about 0.176 to 0.178% (w/w) of the therapeutic formulation. The concentration of beclometasone dipropionate may be selected based on the predetermined dose delivered by the inhaler or spray device and the desired dose of beclometasone dipropionate, the desired dose being between 40 mg and 80 mg. Metered dose inhalers typically deliver a predetermined dose that measures between 25 and 100 μL.

In embodiments where the active agent comprises budenoside, budenoside may comprise about 0.173 to 0.176% (w/w) of the therapeutic formulation. The concentration of budenoside may be selected based on the predetermined dose delivered by the inhaler or spray device and the desired dose of budenoside, the desired dose being between 0.25 mg and 100 mg. Metered dose inhalers typically deliver a predetermined dose that measures between 25 and 100 μL.

In embodiments where the active agent comprises cannabidiol (CBD), CBD may comprise about 3.4802% (w/w) of the therapeutic formulation. The concentration of CBD may be selected based on the predetermined dose delivered by the inhaler or spray device and the desired dose of CBD, the desired dose being between 0.25 mg and 40 mg. Metered dose inhalers typically deliver a predetermined dose that measures between 25 and 100 μL. The CBD may comprise an isolate or extract obtained from Cannabis sp.

The therapeutic formulation further comprises a propellant. In particular non-limiting embodiments, the propellant is a hydrofluoroalkane (HFA). The propellant can include, but is not limited to, HFA-132, HFA 1234ze, HFA 227ea, HFA-152a (Mexichem Fluor; North York, Canada), or combinations thereof. The propellant may comprise between 81% and 98% (w/w) of the therapeutic formulation.

The therapeutic formulation further comprises a co-solvent. The co-solvent is preferably a dehydrated alcohol such as ethanol (anhydrous), but other examples of co-solvents include non-dehydrated alcohols, water, glycerin, propylene glycol, polyethylene glycol, and isopropyl myristate. The co-solvent improves solubility of the active agent, but there is a trade-off with aerosolization. The relative amount of co-solvent should be selected to improve solubility of the active agent in the propellant without decreasing the ability of the inhaler or spray device to aerosolize the therapeutic formulation. The co-solvent may comprise about 0% to about 50% (w/w) of the therapeutic formulation and preferably about 0 to 15% (w/w).

The therapeutic formulation may further comprise a flavouring agent to improve the flavour of the therapeutic formulation or disguise the taste of other components of the therapeutic formulation, thereby enhancing user experience and increasing patient compliance. Examples of suitable flavouring agents include saccharin, (+)Limonene, menthol, citric acid (anhydrous), saccharin sodium dehydrate, sodium citrate, combinations thereof and the like. In some examples, the flavouring agent includes a flavour masking agent. The flavouring agent may comprise about 0% to about 3% (w/w) of the therapeutic formulation, and preferably about 0 to about 1% (w/w).

The therapeutic formulation may further comprise an aromatic agent to improve the odor of the therapeutic formulation or to disguise the odor of other components of the therapeutic formulation, thereby enhancing user experience and increasing patient compliance. Examples of suitable aromatic agents include terpenes.

The therapeutic formulation may further comprise a numbing agent to reduce discomfort associated with inhaling the therapeutic formulation. In particular, inhalation of the alcohol co-solvent may cause irritation to the throat and lungs. Suitable numbing agents may include lidocaine and tetracaine, but the numbing agent is not particularly limited. The numbing agent may comprise about 0 to about 10% (w/w) of the therapeutic formulation, and preferably about 0% to about 5% (w/w).

The therapeutic formulation may further comprise a non-volatile additive. The non-volatile additive may be a co-solvent or solubility agent. In some examples, the non-volatile additive is glycerol. The non-volatile additive may comprise about 0 to 10% (w/w) of the therapeutic formulation, and preferably 0 to 2% of the therapeutic formulation.

The therapeutic formulation may further comprise a solubilizer to improve solubility. Examples of suitable solubilizers include benzalkonium chloride, lecithin (Soya), and sorbitan trioleate (Span® 85). The solubilizer may comprise about 0 to 50% (w/w) of the therapeutic formulation, and preferably about 0 to 3% (w/w) of the therapeutic formulation. The solubilizer may improve stability of the therapeutic formulation.

The therapeutic formulation may further comprise an antioxidant. Examples of suitable antioxidants include acetone sodium bisulfate and ascorbic acid, but the antioxidant is not particularly limited. The antioxidant may comprise about 0 to 5% (w/w) of the therapeutic formulation, and preferably about 0 to 1% (w/w) of the therapeutic formulation.

The therapeutic formulation may further comprise a chelating agent to improve stability and shelf-life of the active agent. The chelating agent may comprise about 0 to 5% (w/w) of the therapeutic formulation, and preferably about 0 to 1% (w/w) of the therapeutic formulation. Examples of suitable chelating agents include edetate sodium/edetate disodium, sodium citrate, and phosphoric acid, but the chelating agent is not particularly limited.

The therapeutic formulation may further comprise a surfactant to reduce the surface tension of the therapeutic formulation. The surfactant may comprise about 0 to 5% (w/w) of the therapeutic formulation, and preferably about 0 to 1% (w/w) of the therapeutic formulation.

The therapeutic formulation may further comprise a preservative to improve the shelf-life of the therapeutic formulation. Examples of suitable preservatives include ammonia, benzalkonium chloride, cetylpyridinium chloride, chlorobutanol, glycerin, methylparaben, propylene glycol, propylparaben, sodium bisulfite, sodium metabisulfite, sodium sulfite, and thymol, but the preservative is not particularly limited. In examples where the preservative includes propylene glycol, the propylene glycol may further act as an anti-nucleating agent. In particular, the propylene glycol may prevent nucleation when cycling between high and lower temperatures.

The therapeutic formulation may further comprise a pH adjuster to improve stability and shelf life of the active agent. Examples of suitable pH adjusters include Hydrochloric acid, Nitric acid, Sodium bisulfate, Sodium hydroxide, Sulfuric acid, and Tromethamine, but the pH adjuster is not particularly limited.

The therapeutic formulation may further comprise an emulsifier. Examples of suitable emulsifiers include cetylpyridinium chloride, oleic acid, and sorbitan trioleate (Span® 85). The emulsifier may improve stability of the therapeutic formulation

The therapeutic formulation may further comprise a dispersant. Examples of suitable dispersants include lecithin (soya), magnesium stearate, oleic acid, polyethylene glycol 1000, and sorbitan trioleate (Span® 85). The dispersant may improve stability of the therapeutic formulation

The therapeutic formulation may further comprise a suspending aid. Examples of suitable suspending aids include polysorbate 80, and polyvinylpyrrolidone K25. The suspending aid may improve stability of the therapeutic formulation

The therapeutic formulation may further comprise a tonicity agent. Examples of suitable tonicity agents include glycerin, sodium chloride, and sodium sulfate (anhydrous). The tonicity agent may improve stability of the therapeutic formulation.

In a particular, non-limiting embodiment, the therapeutic formulation comprises 95.92±9% (w/w) HFA 152a, 1.70±0.2% (w/w) ethanol, 0.41±0.04% (w/w) water, 0.74±0.07% (w/w) nicotine, 0.77±0.08% (w/w) menthol, and 0.46±0.05% (w/w) flavouring agent.

In another, non-limiting embodiment, the therapeutic formulation comprises 0.056% (w/w) nicotine, 1.688% (w/w) propylene glycol, 0.008% (w/w) levomenthol, 0.113% (w/w), 0.-009% (w/w) saccharin, and 98.125% (w/w) HFA 134a.

In another, non-limiting embodiment, the therapeutic formulation comprises 6±0.6% (w/w) ethanol, 0.002±0.0002% (w/w) melatonin, and HFA 152a.

In a further, non-limiting embodiment, the therapeutic formulation comprises 0.1676% (w/w) nicotine, 5% (w/w) ethanol, 0.1% (w/w) glycerol, 0.04% (w/w) saccharin, and 94.6924% HFA 134a.

In a further, non-limiting embodiment, the therapeutic formulation comprises 0.2240% (w/w) nicotine, 5% (w/w) ethanol, 0.1% (w/w) glycerol, 0.04% (w/w) saccharin, and 94.6924% HFA 152a.

In a further, non-limiting embodiment, the therapeutic formulation includes 94.69±0.5% hydrofluoroalkane 152a by weight of the formulation, 5±1% ethanol by weight of the formulation, 0.1±0.1% glycerol by weight of the formulation, 0.04±0.05% saccharin by weight of the formulation, and 0.224%±0.3 nicotine by weight of the formulation.

In a further, non-limiting embodiment, the therapeutic formulation includes 81.5±3% hydrofluoroalkane 152a by weight of the formulation, 15±1% ethanol by weight of the formulation, 2.2±1% water by weight of the formulation, and 1.3±1% caffeine by weight of the formulation.

In a further, non-limiting embodiment, the therapeutic formulation includes 91.5±1% hydrofluoroalkane 152a by weight of the formulation, 8±3.0% ethanol by weight of the formulation, and 0.449±0.5% melatonin by weight of the formulation.

In a further, non-limiting embodiment, the therapeutic formulation includes 89±5% hydrofluoroalkane 152a by weight of the formulation, 2.34±2.5% ethanol by weight of the formulation, 7.3±3% cannabidiol by weight of the formulation, 0.06±0.05% saccharin by weight of the formulation, and 0.84%±0.3 flavoring agents by weight of the formulation.

The therapeutic formulation may be selected for delivery in a conventional metered dose inhaler or a pressurized metered dose inhaler as described herein.

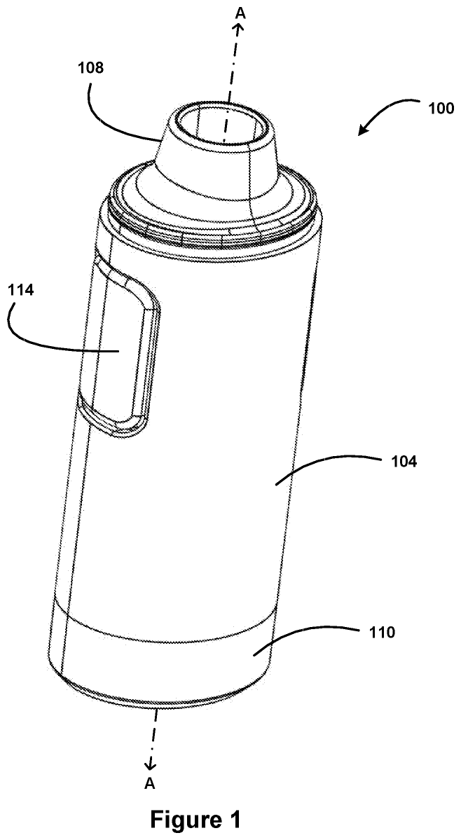

FIG. 1 illustrates an exemplary metered dose inhaler 100 for dispensing a therapeutic formulation from a canister as an aerosol (also referred to herein as an “aerosolized therapeutic formulation”). The metered dose inhaler 100 comprises a housing 104 with a mouthpiece 108. In comparison with the mouthpiece of PCT/CA/2020/051331, the present disclosure provides a shorter and wider mouthpiece that does not interfere with the plumes emitted by the inhaler. The presently described mouthpiece 108 is sized and shaped to conform to the user's mouth without interfering with the exit path of the aerosol. The housing 104 is hollow, in order to receive a pressurized canister of a therapeutic formulation. In some examples, at least the mouthpiece 108 or the bottom end cap 110 is removable from the housing 104 for insertion of the therapeutic formulation. In some examples, at least one of the mouthpiece 108 or the bottom end cap 110 is integral with the housing 104. The housing 104 further includes one or more actuation buttons 114, which, when activated, cause the metered dose inhaler 100 to dispense a pre-determined amount of the therapeutic formulation. In particular examples, the pre-determined amount is between 25 and 100 μL.

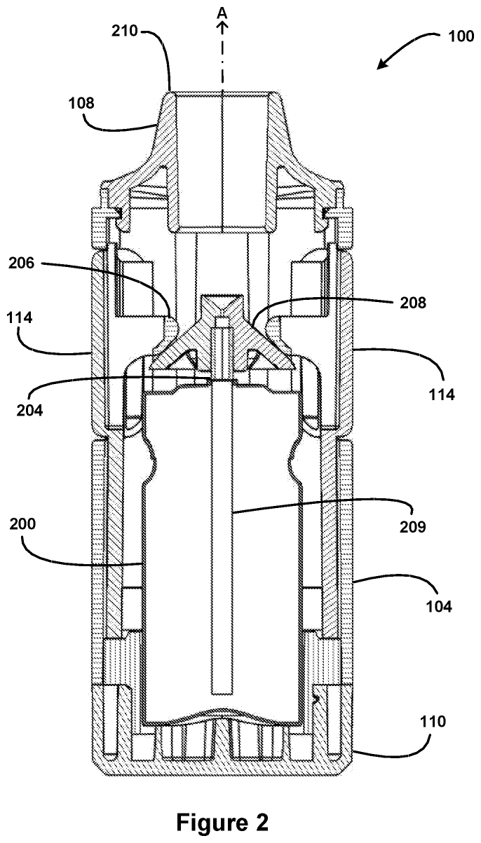

A longitudinal cross-section of the metered dose inhaler 100 is shown in FIG. 2. The housing 104 is configured to receive the canister 200, which stores the therapeutic formulation. The process of filling the canister 200 with a volume of therapeutic formulation, may be conducted at a pressure higher than atmospheric pressure, such that the therapeutic formulation is pressurized. The metered dose inhaler 100 further includes two actuation buttons 114 operably connected to engagement protrusions 206 to depress a stem block 208. The stem block 208 sits on the canister 200 which includes a metering valve assembly. When the actuation buttons 114 are activated or pressed laterally or perpendicular to axis A of the metered dose inhaler 100, the stem block 208 activates the metering valve assembly and releases the therapeutic formulation out of the mouthpiece 108 of the metered dose inhaler 100.

Canister 200 may include a dip tube 209 for conveying the therapeutic formulation from the canister 200 to a metering valve assembly, which includes a hollow dispensing member or valve stem 204. The end of the dip tube 209 may be weighted so that gravity directs the weighted end into the therapeutic formulation, even when the metered dose inhaler 100 is held at an angle. The weighted end may reduce the likelihood that the dip tube conveys air towards the valve stem 204. The metering valve assembly is mounted so as to allow the valve stem 204 to slide relative to the canister 200 between an extended position, to which the valve stem is guided by a biasing mechanism (not shown) in the valve assembly, and a depressed position. Movement of the valve stem 204 from the extended position to the depressed position results in a dose of the therapeutic formulation being dispensed from the canister 200.

The stem block 208 is configured to engage both the engagement protrusions 206 of the actuation buttons 114 and the valve stem 204 of the canister.

In the extended position of the valve stem 204, the pressurized therapeutic formulation in the canister 200 is placed in fluid communication with the metering chamber through the valve stem 204 so that the metering chamber is filled with pressurized therapeutic formulation.

When the valve stem 204 is depressed, the pressurized therapeutic formulation in the metering chamber is isolated from the canister 200 and placed in fluid communication with the external environment through the mouthpiece 108 via the stem block 208. Thus, the volume of pressurized therapeutic formulation in the metering chamber (which includes a metered amount of the therapeutic formulation) is discharged into the external environment via the valve stem 204, then the stem block 208 and ultimately the mouthpiece 108. The user inhales the contents of the mouthpiece 108, thus delivering the aerosolized therapeutic formulation into the user's mouth.

The stem block 208 may be distanced from the mouthpiece 108. The distance between the stem block 208 and the mouthpiece 108 may be selected to control the momentum of the droplets as the droplets enter the user's oropharyngeal region. At lower speeds, oropharyngeal deposition is less likely.

Deposition may also be reduced by selecting suitable dimensions for the mouthpiece 108. Mouthpieces with larger inner diameters can reduce the speed of the droplets exiting the mouthpiece 108, while mouthpieces with narrower inner diameters can interfere with the movement of the plume. Similarly, mouthpieces with longer lengths can interfere with the movement of the plume. The distance from the stem block 208 to an upper end 210 of the mouthpiece 108 may be about 21 to 22 mm when the stem block 208 is in the extended position. When the stem block 208 is in the depressed position, the distance may be about 22.5 to 23.5 mm.

The stem block 208 of the present disclosure is suitable for use in inhalers where the canister 200 is axially aligned with the mouthpiece. In contrast, traditional inhalers include a mouthpiece which is angled away from the longitudinal axis of the canister. Weakness or stiffness in the fingers and hands, particularly in individuals with arthritis and hand size impacts the ability to use traditional metered dose inhalers.

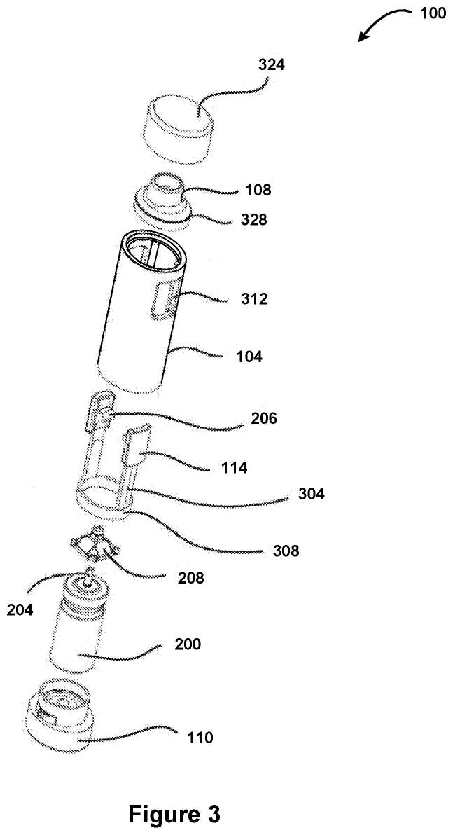

FIG. 3 shows an exploded view of the inhaler. The actuation mechanism includes two actuation buttons 114 each having a button face that is contact surface for the user's digit, a stem block engagement element shown as engagement protrusions 206, and an elongated resilience or spring member 304, which can be flexible plastic member, a flexible metal member, a metal coil, a plastic coil, or the like. The actuation button 114 can be of unitary construction or formed from two or more components, for example, the stem block engagement element and resilience or spring member can be a single piece thereby providing for the use of a different material for the button face. In some embodiments, the stem block engagement element is an engagement protrusion 206.

The resilience or spring member 304 is operatively attached to the actuation button 114 and the engagement protrusion 206 to return the button and the engagement protrusion 206 to their resting positions after release. At a first end, the resilience or spring member 304 is contiguous with or connected to the engagement protrusions 206. The second end of the resilience or spring member 304 is attached either to a band 308 as illustrated in FIG. 3 or a holder for the canister 200.

The engagement protrusions 206, are shaped so that when the button is pressed, the engagement protrusion 206 moves along the stem block 208, thereby pushing the stem block down.

The housing 104 includes openings 312 for the actuation buttons 114. The openings 312 for the actuation buttons 114 are generally in diametrically opposite locations or on opposite sides of the housing 104. The size and configuration of the openings 312 is generally dependent on size and configuration of the actuation buttons 114. Optionally, when inserted into the housing 104, the actuation button 114 surfaces are flush with the surface of the housing 104.

In other examples, the openings 312 in the housing and the actuation buttons 114 can be replaced with pre-formed actuation buttons that are part of the housing 104. The perimeter or edges of each of the buttons being configured to be resiliently flexible so that after the buttons are pressed and released, they return to their starting position.

The mouthpiece 108 may be covered with a mouthpiece cover 324. The mouthpiece 108 and bottom end cap 110 may be threadably connected or snapped to the housing 104. The mouthpiece 108 may include a seal 328 for sealing the mouthpiece to a mouthpiece cover.

The mouthpiece 108 is connected to the housing 104 and the actuation buttons 114 and associated mechanism are placed inside the housing 104. The stem block 208 is connected to the metered canister and together they are inserted into the housing and held in place by a bottom end cap 110, which is removably attached to the housing 104.

In other examples, the canister 200 is inserted into a holder (not shown) which is then inserted into the housing.

The canister 200 may be replaceable by the user such that the inhaler can be reused after the therapeutic formulation in the canister 200 is depleted. Once the canister 200 is spent, the user may remove the empty canister from the housing 104 and replace the empty canister with a full canister.

During use, when pressing the actuation buttons 114 simultaneously, engagement protrusions 206 will move along the top surfaces of stem block 208 at a predetermined angle to cause the valve stem 204 to travel from the extended position to the depressed position along the longitudinal axis A of the metered dose inhaler 100 which activates the metered-dose canister valve. After one activated spray has been performed, a valve spring (not shown) will return the stem block 208 to the extended position. The actuation buttons 114 return to their original predetermined position once the pressure is removed from the actuation buttons 114.

The linear internal design and configuration of the stem block 208 coupled in linear fluid communication with the valve stem 204 permits the pressurized therapeutic formulation from the canister 200 to exit from the mouthpiece 108 of the metered dose inhaler 100 in a linear direction along longitudinal axis C of the stem block 208.

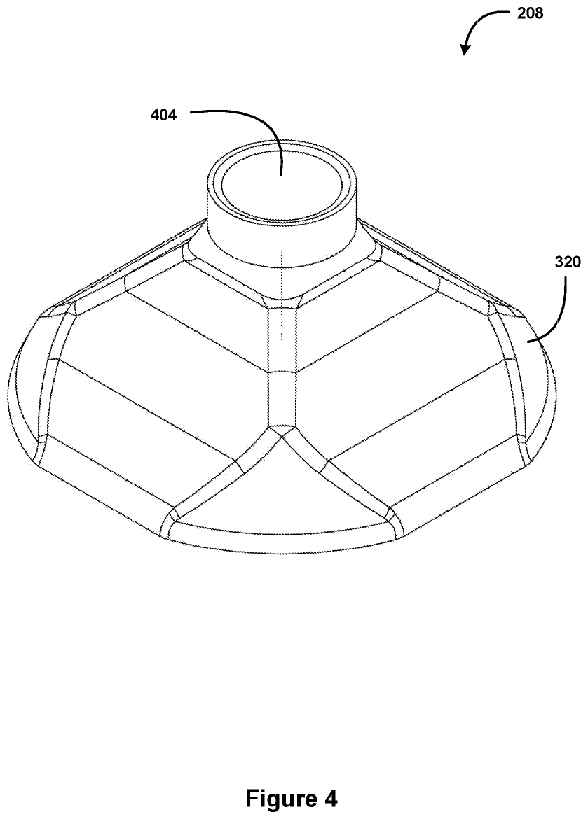

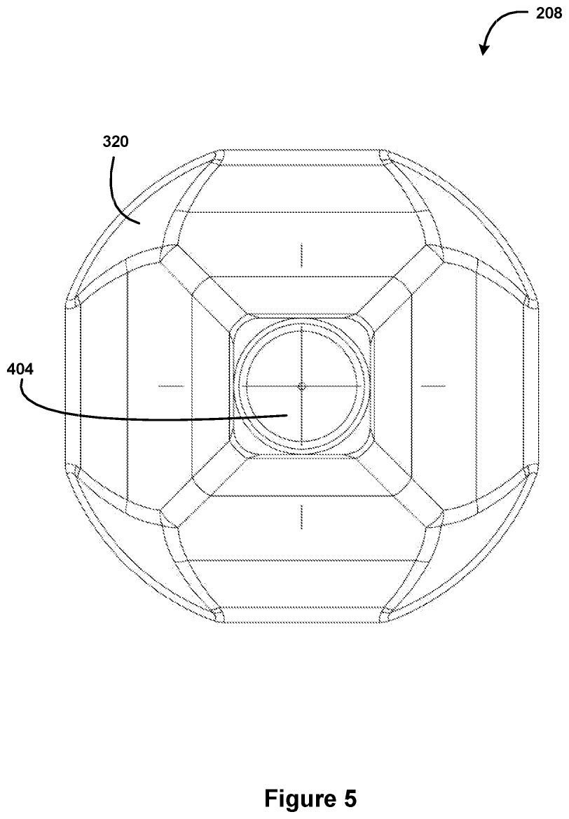

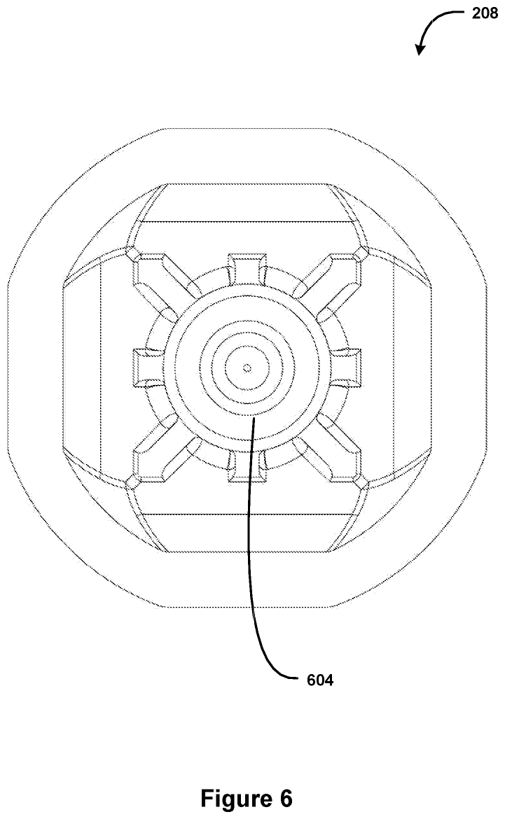

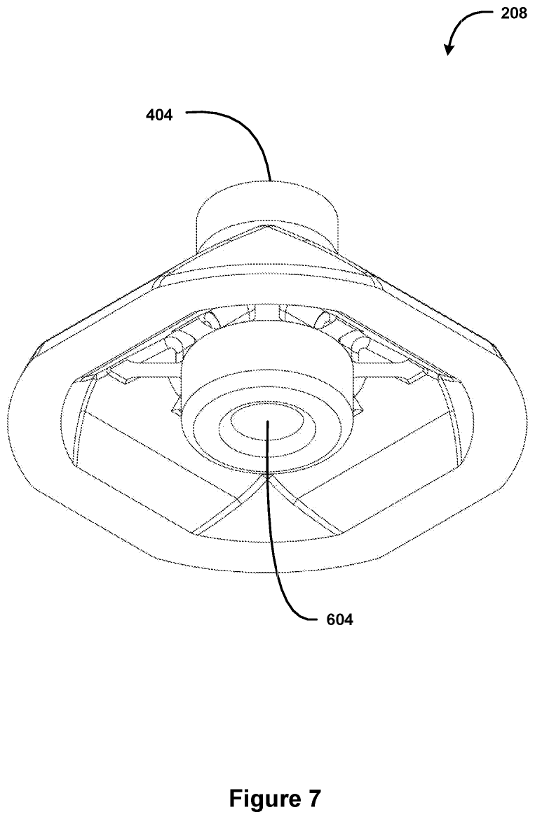

FIGS. 4-8 show the external geometry of the stem block 208. As shown in FIG. 4, the stem block 208 may include a conical outlet 404 for directing the aerosol towards the mouthpiece 108. As shown in FIG. 6, the stem block 208 further includes an inlet 604 for receiving the hollow dispensing member or valve stem 204. The stem block 208 may comprise any suitable material such as aluminum, a polymer, or the like. In specific examples, the stem block 208 comprises acrylonitrile butadiene styrene (Lavergne: Montreal, Canada).

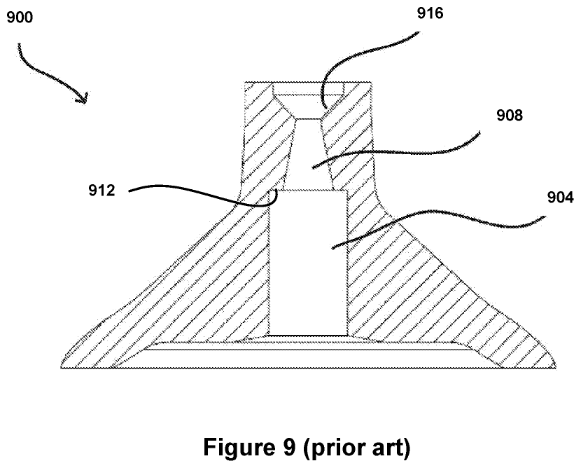

FIG. 9 shows a cross-section of a prior art stem block 900 as disclosed in US patent application entitled “INHALER” having application number PCT/CA/2020/051331, filed 2 Oct. 2020, which is incorporated by reference herein. The prior art stem block 900 includes a valve stem passage 904 sized to receive the hollow dispensing member or valve stem 204 of a metered dose valve. The valve stem passage 904 is fluidly connected to a narrower dispensing passage 908. The lower end of dispensing passage 908 has a width that is narrower than the upper end of valve stem passage 904 to form a ledge 912 at the intersection of the valve stem passage 904 and dispensing passage 908 to prevent upward axial movement of the stem valve into the valve stem passage 904. In this example, the dispensing passage 908 narrows before expanding into a spray nozzle 916.

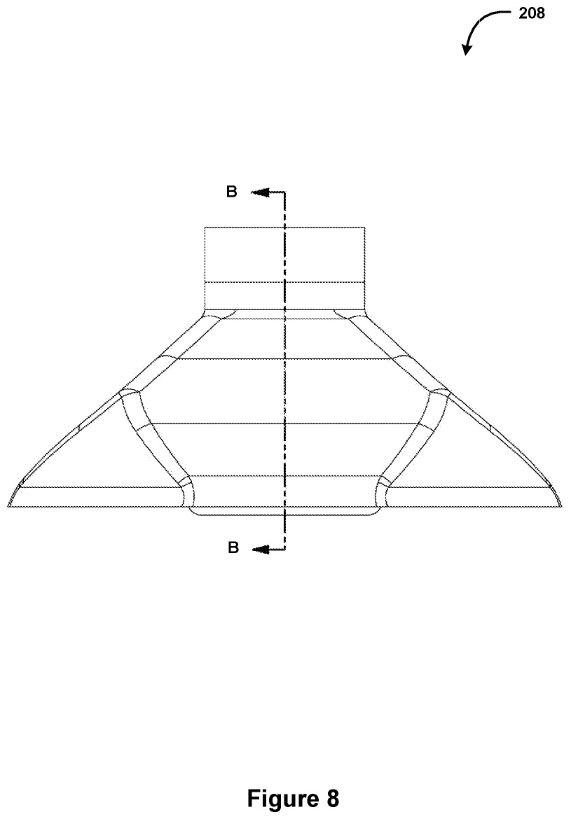

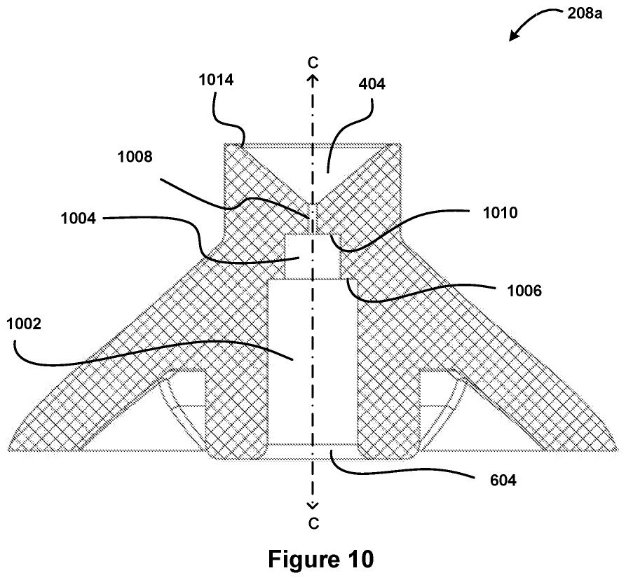

An improved stem block 208a is shown in FIG. 10. FIG. 10 is a cross-section of the stem block 208a of FIG. 8 at plane B. The stem block 208a includes a valve stem passage 1002 sized and dimensioned to receive the hollow dispensing member or valve stem 204 of a metered dose valve through a lower end of the valve stem passage 1002. The valve stem passage 1002 has a diameter that is wider or equally as wide as the outer diameter of the valve stem 204. A close fit between the valve stem passage 1002 and the corresponding valve stem 204 may prevent leakage of the aerosol between the valve stem and the valve stem passage 1002. In one example, the close fit between the valve stem passage 1002 and the corresponding valve stem 204 is about 0.1 mm.

A narrower sump cavity or expansion chamber 1004 is fluidly connected to the valve stem passage 1002 at an upper end, opposite the inlet 604. The lower end of expansion chamber 1004 has a diameter that is narrower than the upper end of valve stem passage 1002 to form a first ledge portion 1006 at the intersection of the valve stem passage 1002 and the expansion chamber 1004. The first ledge portion 1006 is configured to contact an upper end of the valve stem 204 to prevent upward axial movement of the valve stem 204 into the expansion chamber 1004. Therefore, the inner diameter of the expansion chamber 1004 is smaller than the outer diameter of the valve stem 204. In the example shown in FIG. 10, the expansion chamber is 1.5±0.2 mm in length and the diameter is 1.85±0.2 mm, however the dimensions are not particularly limited. The length of the expansion chamber may be between 0 and 10 mm and advantageously between 0 mm and 7 mm. The diameter of the expansion chamber may be between 0.5 mm and 3 mm and advantageously between 1.5 mm and 2.5 mm.

When the valve stem 204 is inserted into the valve stem passage 1002, the valve stem 204 is fluidly connected with the expansion chamber 1004. In some examples, the inner diameter of the expansion chamber 1004 is approximately the same as the inner diameter of the valve stem 204. When the valve assembly is actuated, the expansion chamber 1004 receives the therapeutic formulation from the valve stem 204. Due to propellant evaporation, the therapeutic formulation atomizes within the expansion chamber 1004 to form discrete droplets.

In the example shown, the walls of the expansion chamber are straight and parallel with the longitudinal axis C, however, in other non-limiting examples, the walls of the expansion chamber are angled. In yet further non-limiting examples, the walls of the expansion chamber are curved. In further non-limiting examples, the upper end of the expansion chamber 1004 curves inwardly.

An orifice 1008 is fluidly connected to the expansion chamber 1004 at an upper end, opposite the valve stem passage 1002. The lower end of the orifice 1008 has a diameter that is narrower than the upper end of the expansion chamber 1004 to form a second ledge portion 1010 at the intersection of the expansion chamber 1004 and the orifice 1008. The walls of the expansion chamber 1004 may meet the second ledge portion 1010 at about 90°. The second ledge portion 1006 encourages recirculation of the droplets within the expansion chamber 1004, causing the droplets to deform into aerosol particles. Therefore, the dimensions of the orifice 1008 are selected to control the fine particle fraction of the aerosol dispensed from the metered dose inhaler 100. Generally, the length of the orifice 1008 is between 0.5 mm and 1.5 mm, however in non-limiting examples, the length of the orifice 1008 is between 0.3±0.02 mm and 2±0.2 mm. The diameter of the orifice 1008 may be selected according to the therapeutic formulation. Typically, the diameter of the orifice 1008 is between 0.3 and 0.45 mm, however in non-limiting examples, the diameter of the orifice 1008 is between 0.05 mm and 0.75 mm.

In the embodiment shown in FIG. 10, the orifice 1008 is 1±0.2 mm long and 0.3±0.02 mm in diameter, however the orifice is not particularly limited. As shown in FIG. 10, the inner diameter of the orifice 1008 may be consistent along its length, however in other examples, the inner diameter of the orifice 1008 varies along its length.

The orifice 1008 receives the aerosol from the expansion chamber 1004 and conveys the aerosol through the conical outlet 404. The lower end of the conical outlet 404 is fluidly connected to an upper end of the orifice 1008, opposite the expansion chamber 1004. At the lower end of the conical outlet 404, the conical outlet 404 has the same inner diameter as the upper end of the orifice 1008. At the upper end of the conical outlet 404 which connects to the outside of the stem block 208, the inner diameter of the conical outlet 404 is wider than the inner diameter of the orifice 1008. In the example shown in FIG. 10, the upper end of the conical outlet 404 is 5±0.2 mm in diameter, however the conical outlet 404 is not particularly limited. In other examples, the upper end of the conical outlet 404 is between 3 and 7 mm in diameter. The walls of the conical outlet 404 may range from about 30 degrees to about 60 degrees from the longitudinal axis C. In the example shown in FIG. 10, the walls of the conical outlet 404 are angled at 50±2 degrees from the longitudinal axis. As shown in FIG. 10, the upper end of the conical outlet 404 may include an outwardly curved edge 1014 to improve performance. The length of the outwardly curved edge 1014 may measure between 0.2 mm and 1.0 mm. In the example shown in FIG. 10, the length of the outwardly curved edge 1014 is 0.5±0.1 mm. The conical outlet 404 conveys the aerosol from the stem block 208. The outlet 404 may further direct the aerosol towards the mouthpiece 108 and into the user's mouth. In some embodiments, the aerosol is inhaled (pulmonary delivery) and in other embodiments, the aerosol is administered under the user's tongue (sublingual delivery).

Each of the valve stem passage 1002, the expansion chamber 1004, the orifice 1008, and the conical outlet 404, are axially aligned. As shown in FIG. 10, the valve stem passage 1002, expansion chamber 1004, orifice 1008 and conical outlet 404 share a common longitudinal axis: axis C. The stem block 208a may be axially aligned with the inhaler 100. In particular examples, axis A of inhaler 100 corresponds with axis C of stem block 208. In these examples, the valve stem passage 1002, the expansion chamber 1004, the orifice 1008, and the conical outlet 404, are axially aligned with the canister 200.

The stem block 208a may comprise any suitable material including, but not limited to, metals, metal alloys, polymer (including synthetic polymers), resin, and combinations thereof.

In examples where stem block 208a comprises a polymer, the stem block 208a may be manufactured by injection molding. The stem block 208a overcomes several of the limitations of injection molding of MDIs. Because valve stem passage 1002, expansion chamber 1004, exit orifice 1008, and outlet 404, are axially aligned, exit orifice 1008 may be any length and the stem block 208a may include undercuts. Furthermore, the inner surface of the stem block 208a can be smoother, particularly the transition from the expansion chamber 1004 to the outlet 404.

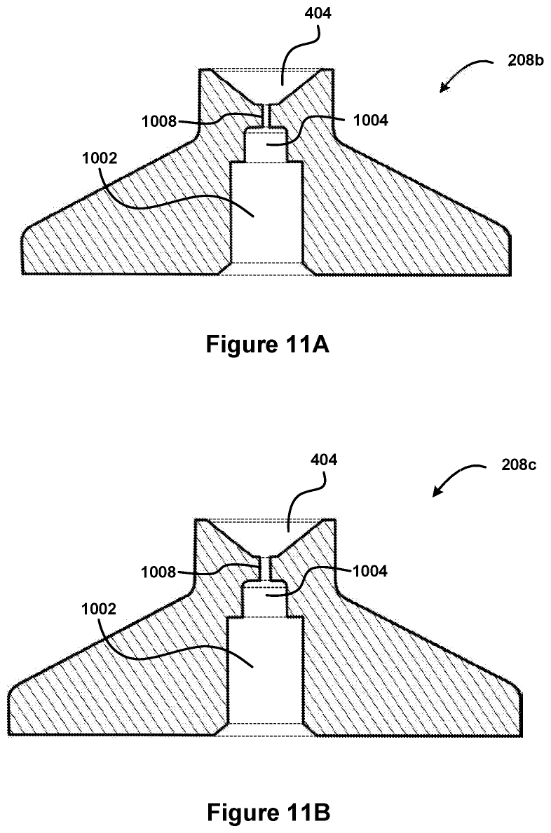

In view of the above, it will now be apparent that variant, combinations, and subsets of the foregoing embodiments are contemplated. For example, variations in the shape and dimensions of the stem block 208a are contemplated. Non-limiting examples of stem block 208b 208c, 208d, 208e, 208f, 208g are shown in FIGS. 11A, 11B, 12A, 12B, 13A, and 13B respectively. Stem blocks 208a, 208b 208c, 208d, 208e, 208f, 208g are generically referred to herein as “stem block 208” or collectively as “stem blocks 208”.

FIG. 11A and FIG. 11B show stem blocks 208b and 208c respectively. Stem block 208a and 208b differ in the diameter of orifice 1008.

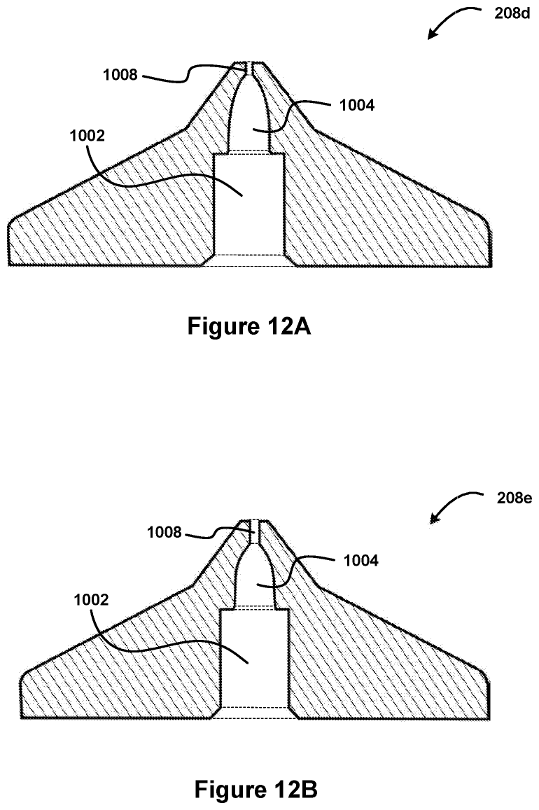

FIG. 12A and FIG. 12B show stem blocks 208d and 208e, respectively. Stem blocks 208d and 208e do not include a conical outlet 404, and the aerosol exits the stem blocks 208d, 208e directly from the orifice 1008. Additionally, the expansion chamber 1004 of the stem blocks 208d and 208e is conically shaped. The expansion chamber 1004 narrows gradually along its length such that the diameter of the expansion chamber 1004 is equal to the diameter of the orifice 1008, where the expansion chamber 1004 connects to the orifice 1008. Thus, the stem blocks 208d and 208e do not include the second ledge portion 1010. In the examples shown in FIGS. 12A and 12B, the expansion chamber 1004 has a convex cone shape, however the expansion chamber 1004 is not particularly limited. In other examples, the expansion chamber 1004 has a concave cone shape. In yet other examples, expansion chamber 1004 may have a straight cone shape. Stem blocks 208d and 208e differ in the length of the expansion chamber 1004 and the length and diameter of the orifice 1008.

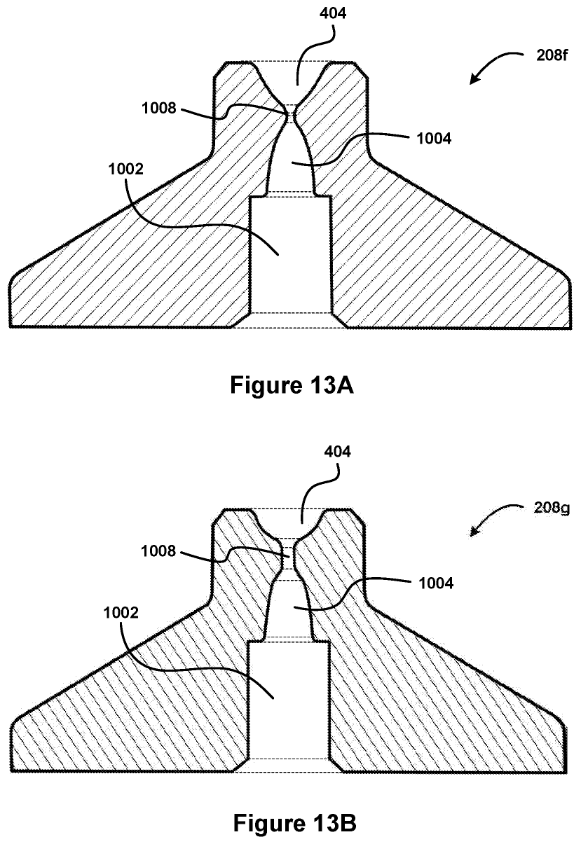

FIG. 13A and FIG. 13B show stem blocks 208f and 208g, respectively. Stem blocks 208f and 208g include expansion chambers 1004 that are conical and outlets 404 that are conical. In these examples, both the expansion chambers 1004 and the conical outlets 404 are convexly conical, however the stem block 208 is not particularly limited. The expansion chamber 1004 may be cylindrical, conical, convexly conical, or concavely conical. The conical outlet 404 (if present) may be straight conical, convexly conical, or concavely conical. Stem blocks 208f and 208g differ in the length and diameter of the orifice 1008.

While stem block 208 has been described as a distinct component of the metered dose inhaler 100, it should be understood that other variations are contemplated. In specific non-limiting examples, stem block 208 is integral with housing 104. In other non-limiting embodiments, stem block 208 is integral to mouthpiece 108. In yet other embodiments, stem block 208 is integral to actuation button 114.

Stem block 208 provides a number of improvements over prior art metered dose inhalers (MDIs) such as the conventional asthma inhaler.

Firstly, stem block 208 is suitable for use in an upright orientation with actuator buttons at the side, instead of at the bottom end. This configuration is easier for users to handle, particularly users with small or weak hands, although other users will naturally benefit from the convenience.

Secondly, because the inhaler is easier to manipulate, drug delivery is improved. Metered dose inhalers must be held in a particular orientation during use to ensure that the aerosol is directed towards the oropharyngeal region. Even slight deviations in the angle will cause the aerosol to impact and deposit on the soft palate or tongue. Therefore, an upright inhaler, which is easier to manipulate, will improve the likelihood that the aerosol is delivered to the lungs.

Thirdly, stem block 208 overcomes the performance limitations of an upright MDI. Inhalers fired “valve up” (with a dip tube) are generally considered less reliable than conventional “valve down” inhalers (without a dip tube). Stem block 208 improves the performance of an upright metered dose inhaler so that drug delivery is superior to a conventional inhaler. As described in the examples below, droplet size and distribution for the stem block 208 meets or exceeds that of a conventional inhaler.

Lastly, the axial alignment of stem block 208 reduces the occurrence of clogs in the MDI. As compared with conventional actuators in which the expansion chamber and the orifice are substantially perpendicular, stem block 208 is axially aligned, which reduces opportunities for the formulation to accumulate within stem block 208.

As described in the examples below, performance of the therapeutic formulation in the inhaler 100 exceeds that of a conventional inhaler.

### EXAMPLES

### 1 Equipment & Materials

The equipment and materials used to generate the data presented in this report are listed below.

### Equipment:

a. Mettler Toledo™ Analytical Balances: XP205, XS204, AT261, XS802S
b. Pamasol™ P2016 Laboratory Plant Crimp and Propellant Filler
c. Pamasol™ P2002 Aerosol Filler and Crimper System
d. Agilent™ 1100 Series HPLC, G1322A Degasser, G1311A QuatPump, G1313AALS, G1316A Col, G1314A VWD
e. Copley Scientific™ Next Generation Impactors
f. MSP™ NGI Leak Tester
g. MSP™ NGI Gentle Rocker 4515
h. Copley Scientific™ HPC5 Pumps
i. Copley Scientific™ TPK2000 Flow Regulator
j. Copley Scientific™ DMF 2000 Mass Flow Meter
k. ELGA PureLab® Prime, PureLab Ultra Genetic Water system
l. Decon™ FS400b Ultrasonic Bath
m. Vindon Scientific™ Temperature and Humidity Controlled Stability Cabinet
n. GenLab™ MINI50/TDIG Temperature Controlled Stability Cabinet
o. Metrohm™ 684 KF Coulometer
p. Rainin™ EDP3-Plus Analytical pipettes

### Materials:

a. Melatonin, batch: SLBZ6359 (Sigma®: Gillingham, England)
b. Beclomethasone dipropionate (BDP), Batch OZID004
c. Budesonide, Batch OZID007
d. Chromasolv™ Methanol (99.9%) (Honeywell: Charlotte, North Carolina, USA)
e. Ethanol, batch 314763 and batch 21J204140 (Scientific Laboratory Supplies Ltd.™: Nottingham, United Kingdom)
f. Purified Water, prepared using Elga PureLab® Ultra system
g. Zephex® HFA 134a, Batch RB20610-3 (Koura: San Luis Potosi, Mexico)
h. Zephex® HFA 152a, UN1030 (Koura: San Luis Potosi, Mexico)

### Metered Dose Inhaler (MDI) Hardware:

a. Actuators
   i. Stem Block 208a: Bespak™ 0.30 mm actuator (ID 277), Batch BK0486487 (North Lynn Industrial Estate, England), 1.0 mm length
   ii. Conventional actuator: Presspart™ 0.30 mm actuator (ID 290), Batch PPT0027735 (Blackburn, United Kingdom), 0.65 mm length
b. Valves:
   i. Bespak™ 50 μl valve for upright use with dip tube, batch BK0760388 (ID: 333 & 346), batch BK0880753 (ID: 364) (North Lynn Industrial Estate, United Kingdom)
   ii. Bespak™ 50 μl valve for inverted use with glass bottle (ID: 320), Batch BK0642138 (North Lynn Industrial Estate, United Kingdom)
   iii. Aptar™ 50 μl valve for inverted use, Batch BE-ECHPI-ASS-2021-66-V1 (ID:330) and Batch SE23-383 (Milton Keynes, United Kingdom)
c. Canister:
   i. Presspart™ C0128-FEP 19 ml canister, Batch PPB0008265 (ID: 240 & 248) (Blackburn, United Kingdom)
d. Glass bottle:
   i. St Gobain™ Type III Glass, 15 ml aerosol tube, batch 711175 (ID: 2) (Leicestershire, United Kingdom)

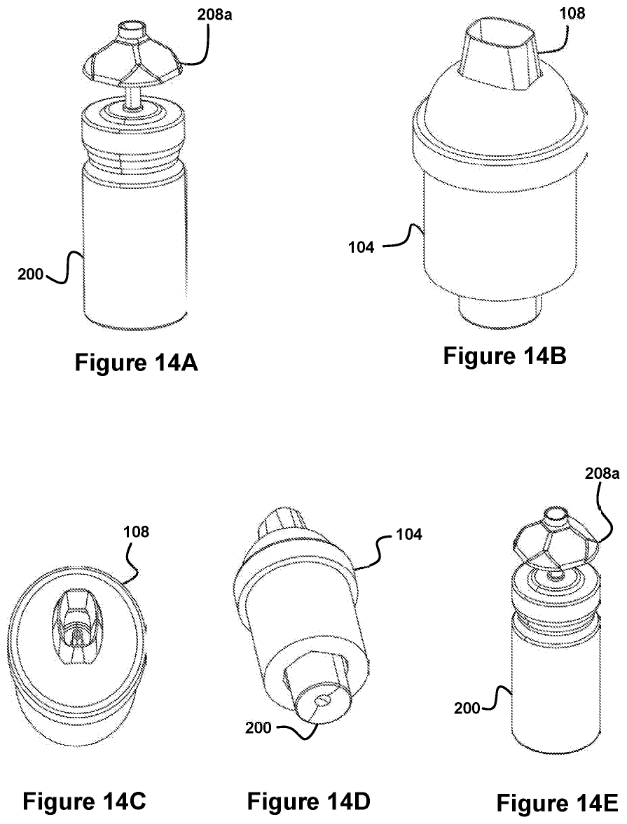

The examples described herein further include stem block 208a, as described according to the present specification and shown in FIGS. 14A to 14E. The stem block 208a used in the examples was manufactured by Presspart™ (Blackburn, United Kingdom). The stem block 208a can be attached to a canister 200 and metering valve with a 3.16 mm valve stem diameter as shown in FIGS. 14A and 14E. To facilitate actuation of the MDI, a housing 104 was fabricated to allow the base of the MDI to be pushed inwards when each dose is fired. The housing 104 includes a mouthpiece 108 as shown in FIGS. 14B and 14C. In the example shown in FIGS. 14A to 14E, the orifice 1008 of the stem block 208a has a diameter of 0.30 mm and a length of 1 mm. The length of the expansion chamber 1004 is 1.5 mm.

### 2 Methodology

All manufacturing tolerances were within 2 percent by mass of target.

Drug residual measurements (total can content) was determined following storage at 40° C. and 75% relative humidity.

Drug delivery was determined in accordance with the United States Pharmacopeia (USP). Drug delivery metrics (metered dose, delivered dose, fine particle dose/fraction, mass median aerodynamic diameter (MMAD) and geometric standard deviation (GSD)) were determined using a Next Generation Impactor (NGI) and dose uniformity sampling apparatus (DUSA). Sampling flow rate was 30 L/min for both NGI and DUSA measurements and data processing was performed using CITDAS V3.10 Software (Copley Scientific™: Colwick, United Kingdom).

Melatonin, Beclomethasone dipropionate (BDP), and Budesonide content within test samples was determined by reverse phase high pressure liquid chromatography (HPLC) with single wavelength ultraviolet detection on an Agilent™ 1100 system running Empower 3 Chromatography Software (Waters, UK).

HPLC assays were established, and system suitability confirmed. A summary of the routine assays used for analyzing test samples is given in Table 1 (Melatonin), Table 2 (BDP), and Table 3 (Budesonide).

TABLE 1
Melatonin Routine Assay Summary

| Parameter/Item | Description | |
|---|---|---|
| Column | Hypersil C18 150 × 4.6 (5 μm) | |
| Mobile Phase | KHPO₄ Buffer:Acetonitrile (70:30) | |
| | Buffer preparation: 0.5 g/L Potassium | |
| | Dihydrogen Phosphate. | |
| | Adjust to pH 3.5 | |
| | using orthophosphoric acid | |
| Flow Rate | 1.3 | mlmin⁻¹ |
| Injection Volume | 20 | μl |
| Column oven temperature | 35° | C. |
| Detection, Wavelength | 222 | nm |
| Chromatograph Run Time | 5 | minutes |
| Expected Drug Retention Time | 2.6 | mins |
| Syringe Wash Solution | Acetonitrile:Water (60:40) | |
| Dilution Solvent | Water:Acetonitrile (75:25) | |
| Linear Range | 0.2-75 | μgml⁻¹ |

| | |
|---|---|
| Vials | Chromatography Direct (amber) |

TABLE 2
Beclomethasone dipropionate (BDP) Routine Assay Summary

| Parameter/Item | Description | |
|---|---|---|
| Column | Modus C18 5 μm (100 × 4.6 mm) MOS | |
| Mobile Phase | Acetonitrile:Water (60:40) | |
| Flow Rate | 2.0 | mlmin⁻¹ |
| Injection Volume | 50 | μl |
| Column oven temperature | 30° | C. |
| Detection, Wavelength | 238 | nm |
| Chromatograph Run Time | 8 | minutes |
| Expected Drug Retention Time | 3.4 | mins |
| Syringe Wash Solution | Acetonitrile:Water (60:40) | |
| Dilution Solvent | Acetonitrile:Water (60:40) | |
| Linear Range | 0.18-78 | μgml⁻¹ |

| | |
|---|---|
| Vials | Chromatography Direct |

TABLE 3
Budesonide Routine Assay Summary

| | |
|---|---|
| Parameter/Item | Description |

| Column | Hypersil C18 150 × 4.6 (5 μm) | |
|---|---|---|
| Mobile Phase | Acetonitrile: Buffer (40:60) | |
| | Buffer Preparation: 3.75 g of disodium | |
| | hydrogen orthophosphate dihydrate into | |
| | 1 L DI Water. | |
| | Adjust to pH 3.2 ± 0.2 with | |
| | orthophosphoric acid | |
| Flow Rate | 1.5 | mlmin⁻¹ |
| Injection Volume | 100 | μl |
| Column oven temperature | 30° | C. |
| Detection, Wavelength | 240 | nm |
| Chromatograph Run Time | 15 | minutes |
| Expected Drug Retention Time | 4.9 mins (r-epimer) & 5.2 mins (s-epimer) | |
| Syringe Wash Solution | Acetonitrile:Water (32:68) | |
| Dilution Solvent | Acetonitrile:Water (32:68) | |
| Linear Range | 0.15-15 | μgml⁻¹ |

| | |
|---|---|
| Vials | Chromatography Direct |

### 4 Example 1: Melatonin

### 4.1 Determination of Solubility/Miscibility (Ethanol Selection)

Solubility of each drug was evaluated in mixtures were investigated in HFA152a and HFA134a propellant.

### 4.2 Quantitative Solubility of Melatonin and Excipients in Ethanol/HFA152a134a Propellent

Supersaturated melatonin formulations were prepared in HFA 152a containing either 0%, 5%, 8%, 10% or 15% w/w ethanol. The upper solubility limit of melatonin was determined by filtering the supersaturated solutions through 0.2 μm polytetrafluoroethylene (PTFE) filters (see Table 4) and quantifying test samples by HPLC.

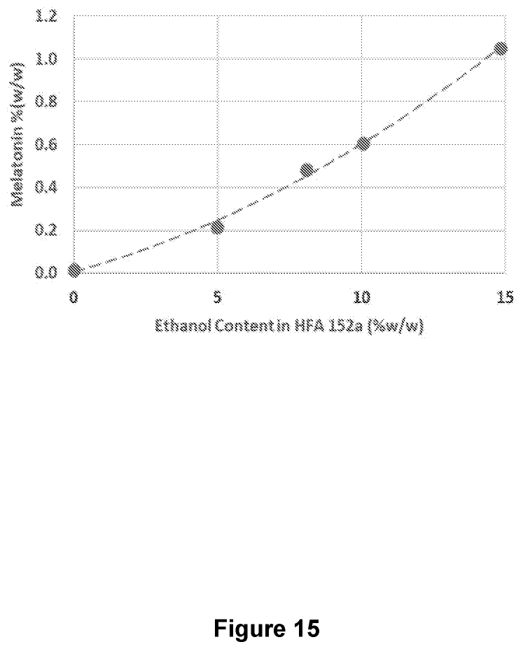

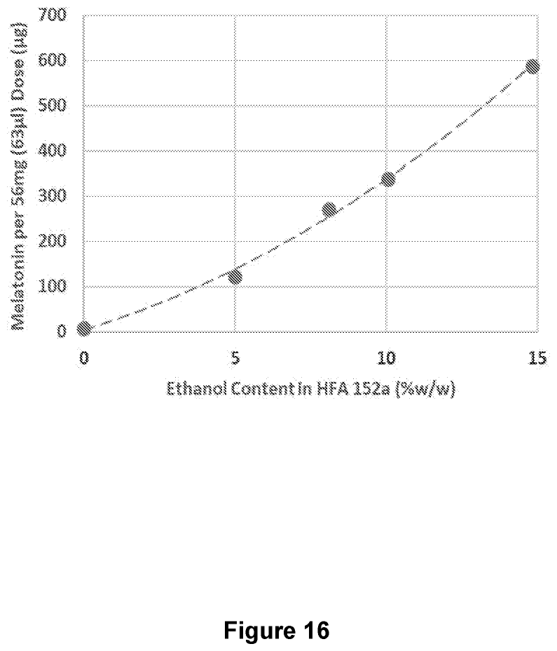

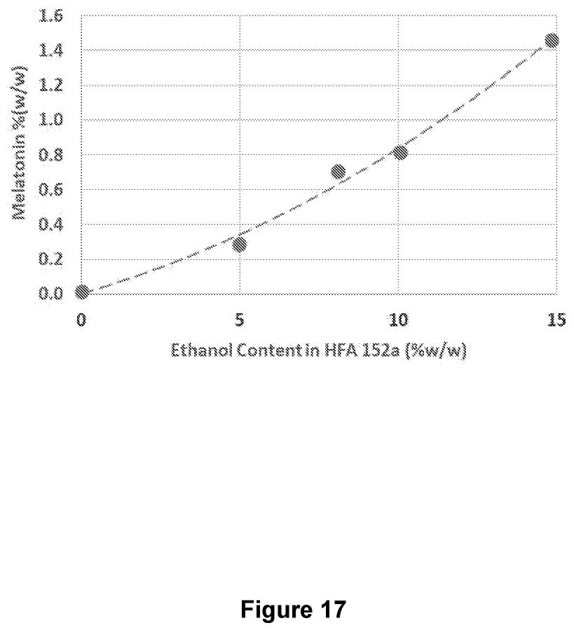

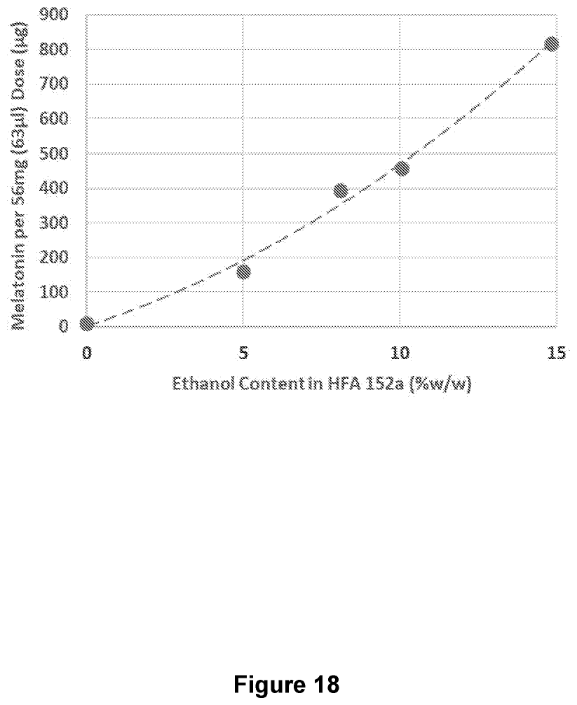

Data was obtained at 5° C. (see FIG. 15 and FIG. 16) and T=20° C. (see FIG. 17 and FIG. 18) and presented as % w/w, μg/50 μl and μg/63 μl (see also Table 4).

TABLE 4
Concentration of Melatonin in filtered supersaturated HFA152a solutions

| | | | Melatonin μg per | | *Melatonin μg per | |
|---|---|---|---|---|---|---|
| Ethanol | Melatonin %(w/w) | | 44 mg (50 μl) | | 56 mg (63 μl) | |
| %9 w/w) | T = 5° C. | T = 20° C. | T = 5° C. | T = 20° C. | T = 5° C. | T = 20° C. |
| 0 | 0.01 | 0.02 | 6 | 7 | 8 | 9 |
| 5 | 0.22 | 0.28 | 95 | 125 | 121 | 159 |
| 8 | 0.48 | 0.70 | 212 | 310 | 270 | 395 |
| 10 | 0.60 | 0.82 | 266 | 359 | 339 | 456 |
| 15 | 1.05 | 1.46 | 462 | 642 | 588 | 817 |

The data presented in Table 4 and FIG. 17 show that 5% w/w ethanol in HFA 152a is suitable for delivering 100 μg of melatonin using either a 50 μl or 63 μl valve. However, a 63 μl dose volume or 6% w/w ethanol is preferrable to ensure solubility is maintained at low temperatures (121 μg melatonin per 63 μl of dose volume was observed for 5% w/w ethanol at 5° C., see Table 4).

Likewise, 8% w/w ethanol in HFA 152a is shown to deliver 200 μg of melatonin in either a 50-μl or 63-μl dose volume (see Table 4). Increasing ethanol further increases the ability to solubilize melatonin, however, increasing ethanol content decreases drug delivery performance, as described later.

FIG. 15 is a graph showing melatonin solubility in ethanol-based HFA 152a formulations at 5° C.

FIG. 16 is a graph showing melatonin solubility in ethanol-based HFA 152a formulations at 5° C.

FIG. 17 is a graph showing melatonin solubility in ethanol-based HFA 152a formulations 20° C.

FIG. 18 is a graph showing melatonin solubility n ethanol-based HFA 152a formulations at 20° C.

When packaged in glass bottles, 100 μg/50 μl (6% w/w ethanol in HFA 152a) and 200 μg/50 μl (8% w/w ethanol in HFA 152a) were observed to be clear solutions at 5° C.

### 4.3 Visual Solubility of BDP and Budesonide HFA 152 Formulations

With regards to the propellant HFA 152a, this report compares the performance of the stem block 208a using 100 μg/50 μl BDP in 6% w/w ethanol and 100 μg/50 μl budesonide in 8% w/w ethanol.

Solubility was confirmed visually (at 5° C. and 20° C.) by packaging each formulation within glass bottles.

### 4.4 Visual Solubility of Melatonin, BDP & Budesonide HFA 134a Formulations

This report also presents the drug delivery performance of the stem block 208a when used with the propellant HFA 134a.

Formulations were prepared containing 100 μg/50 μl of either Melatonin, BDP or Budesonide in 12% w/w ethanol and HFA 134a. Solubility was confirmed visually (at T=5° C. and T=20° C.) by packaging each formulation within glass bottles.

Solubility was confirmed visually (at T=5° C. and T=20° C.) for 50 μg/50 μl melatonin in 5% ethanol and HFA 134a.

### 4.5 Drug Delivery: Melatonin, BDP and Budesonide Formulations

Four studies are presented herein. Section 4.6 demonstrates the drug delivery performance of 100 μg & 500 μg melatonin using HFA 152a as the propellant in a conventional actuator for an MDI. Section 4.7 compares “valve up” and “valve down” actuation. Section 4.8 demonstrates the performance of melatonin, BDP, and Budesonide at a concentration of 100 μg/50 μl using HFA 152a as the propellant. Sections 4.9-4.10 demonstrate the performance of melatonin, BDP, and budesonide at a concentration of 100 μg/50 μl using HFA 134a as the propellant. Section 4.12 demonstrates the performance of melatonin at a concentration of 50 μg/50 μl in 5% w/w ethanol with HFA 134a as the propellant.

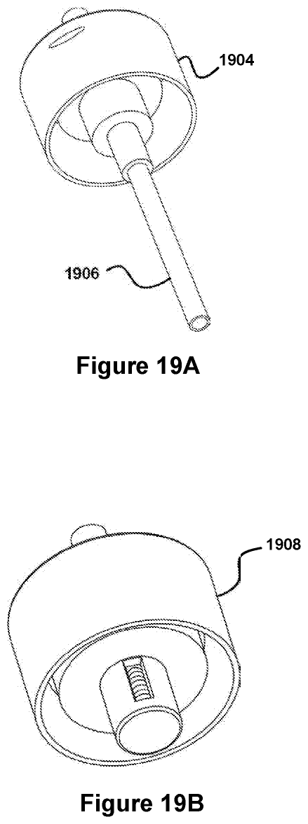

FIG. 19A is a perspective view showing a first metering valve 1904 with a dip tube 1906 and FIG. 19B is a perspective view showing a second metering valve 1908 without a dip tube. At 1904, a Bespak™ 50 μl metering valve is shown with the dip tube 1906 for “valve up” use. At 1908, an Aptar™ 50 μl metering valve without a dip tube is shown for “valve down” use. Metered dose inhalers (MDIs) are conventionally actuated in an upright (or “valve down”) orientation using a metering valve that lacks a dip tube, such as the metering valve 1908 shown in FIG. 19B. The stem block 208a can be used for inverted (or “valve up”) actuation using metering valve 1904 fitted with dip tube 1906.

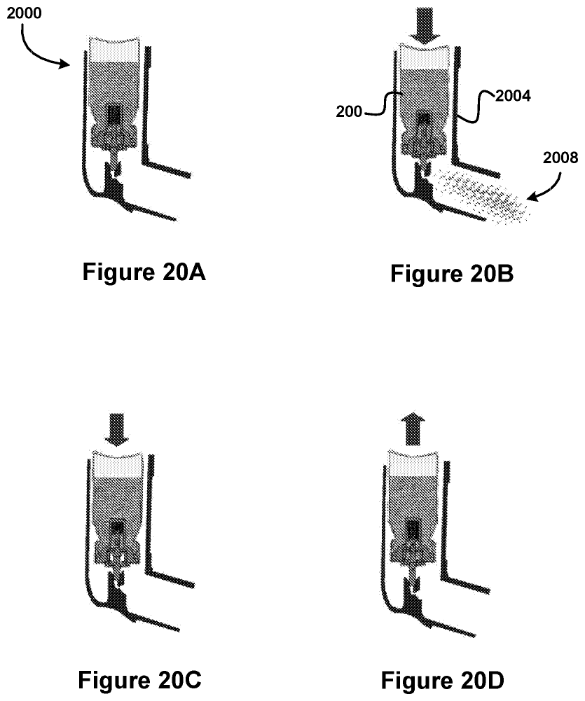

The metering chamber filling sequence for a conventional actuator 2000 with no dip tube is presented in FIGS. 20A to 20D. FIG. 20A shows the conventional actuator 2000 at rest. FIG. 20B shows the canister 200 being pushed into the housing 2004 causing a dose of therapeutic formulation 2008 to be dispensed from the conventional actuator 2000. In FIG. 20C, the metering chamber is empty. FIG. 20D shows the canister 200 being released and the chamber refilling in response. It is possible to fire the conventional actuator 2000 in any orientation, however, the conventional actuator 2000 must be in the correct (“valve down”) orientation when the chamber refills for the next dose. In other words, the metering chamber must refill from the liquid phase of the canister.

In Sections 4.5, 4.6 and 4.7, each inhaler was fired in the intended orientation: “valve down when fitted with metering valve 1908 (Aptar™, without dip tubes); “valve up” when fitted with metering valve 1904 (Bespak™ valves with dip tube 1906).

In Sections 4.8-4.10, where necessary, the tested MDI was rotated to the “filling” orientation after actuation but prior to releasing the valve to its rest position (see FIG. 17). Thus, in Sections 4.8-4.10 it was possible to determine the drug delivery performance of the conventional actuator 2000 fired “valve down”, and “valve up”.

### 4.6 Performance of 100 μg & 500 μg Melatonin with Conventional Actuator

Melatonin loading of 100 μg/50 μl and 500 μg/50 μl was achieved. Both formulations were confirmed visually to be soluble formulations at 4° C. and 20° C.

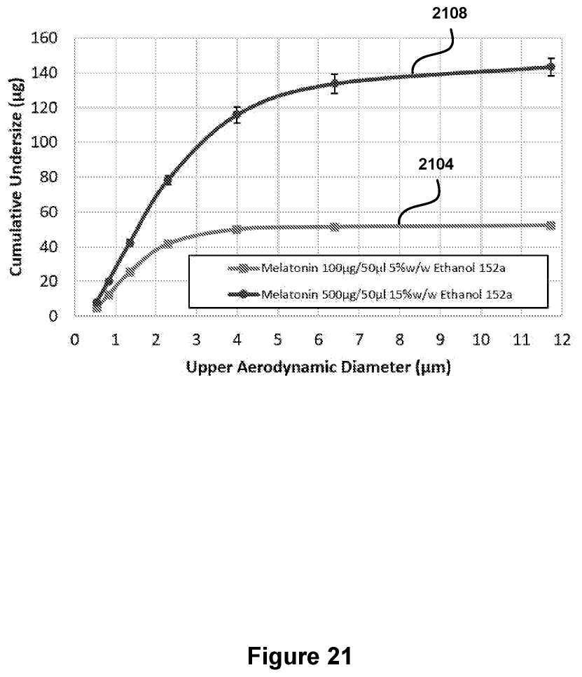

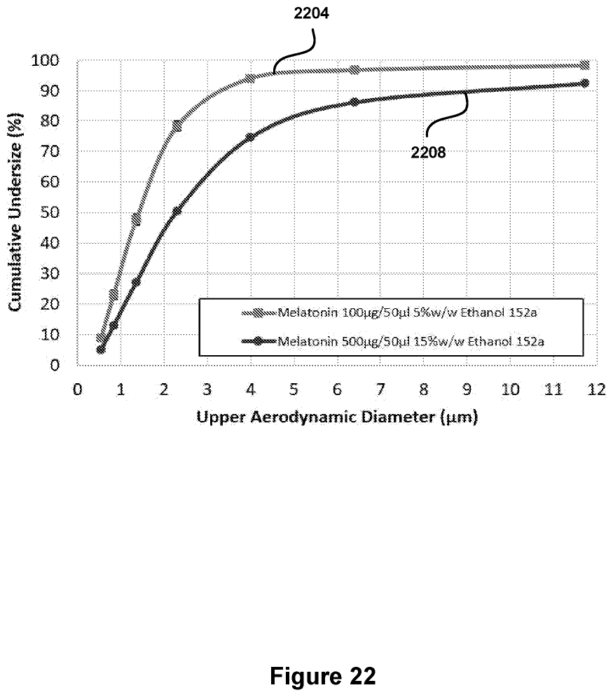

Melatonin drug delivery data is presented in Table 5, FIG. 21, and FIG. 22 for 100 μg/50 μl melatonin (5% w/w Ethanol) and 500 μg/50 μl melatonin (15% w/w Ethanol) using HFA 152a as the propellant. The formulations were delivered using the conventional actuator 2000 (Bespak™, 0.3 mm).

Metered dose values are close to target; 103±4 μg and 499±5 μg respectively. Fine particle dose <5 μm Aerodynamic diameter (FPD) for the 100 μg formulation was 51±1 μg; and Fine Particle Fraction <5 μm Aerodynamic diameter (FPF)=55±2%.

Ethanol was increased to 15% w/w to maintain the melatonin in solution when the dosage was increased to 500 μg/50 μl. The increase of ethanol to 15% w/w had the consequence of reducing atomization efficiency; the fine particle dose for the 500 μg formulation was 125±5 μg; FPF=28±1%.

The MMAD was dependent upon the concentration of non-volatile within the formulation. The MMAD was 1.4±0.1 μm for the 100 μg/50 μl formulation and 2.3±0.1 μm for the 500 μg/50 μl formulation.

TABLE 5
Melatonin Delivery from 100 μg/50 μl (5%
w/w Ethanol) and 500 μg/50 μl (15% w/w
Ethanol) HFA 152a using the conventional actuator (0.30 mm)

Melatonin Dose (μg/50 μl)

| | 100 | 500 |
|---|---|---|
| Total Dose Per Shot (μg) | 103 ± 4 | 499 ± 5 |
| Delivered Dose (μg) | 92 ± 4 | 441 ± 5 |
| Fine Particle Dose (μg) | 51 ± 1 | 125 ± 5 |
| Fine Particle Fraction | 55 ± 2 | 28 ± 1 |
| (FPF) (%) | | |
| MMAD (μm) | 1.4 ± 0.1 | 2.3 ± 0.1 |
| n | 3 | 3 |

FIG. 21 is a graph showing the cumulative undersize (μg) for melatonin delivery. 2104 indicates 100 μg/50 μl melatonin (5% w/w ethanol) and 2108 indicates 500 μg/50 μl melatonin (15% w/w ethanol). HFA 152a was the propellant. Both formulations were delivered with the conventional actuator 2000 (n=3, Mean±standard Deviation).

FIG. 22 is a graph showing the cumulative undersize (%) for melatonin delivery. 2204 indicates 100 μg/50 μl melatonin (5% w/w Ethanol) and 2208 indicates 500 μg/50 μl melatonin (15% w/w ethanol). In both formulations, HFA 152a was the propellant (n=3, Mean±standard deviation).

### 4.7 “Valve Up” Orientation Vs. “Valve Down” Orientation

A comparison was conducted between the conventional actuator 2000 (100 μg) fired “valve down” (no dip tube), the conventional actuator 2000 (100 μg) with dip tube 1906 fired “valve up”, the stem block 208a and housing 104 (100 μg) fired “valve up” (with dip tube 1906), and the stem block 208a and housing 104 (200 μg) fired “valve up” (with dip tube 1906).

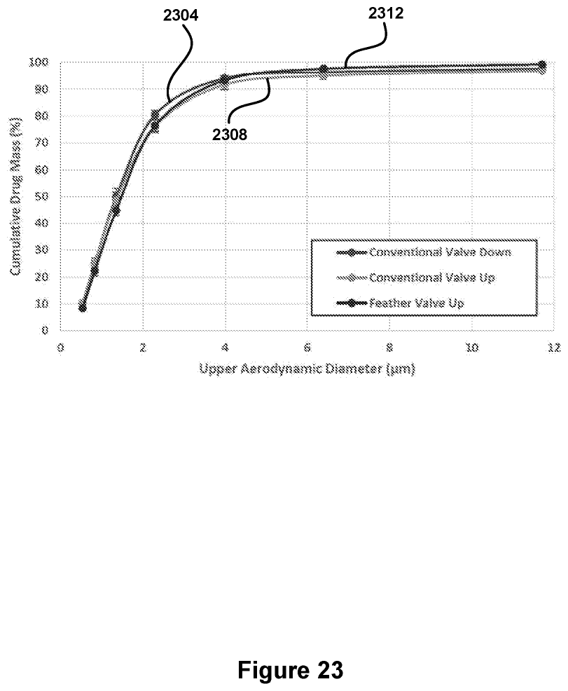

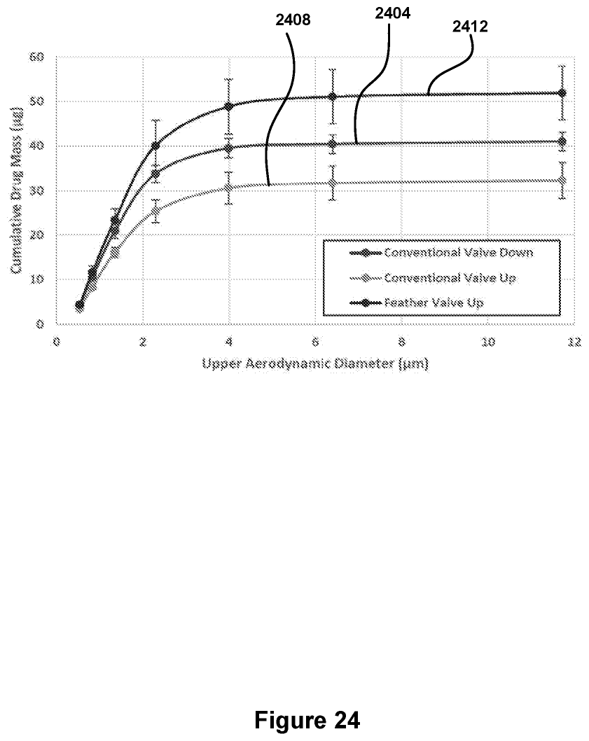

Results obtained are summarized in Table 6, FIG. 23 and FIG. 24.

Table 6 shows that the dip tube 1906 added to the conventional actuator 2000 (as indicated at B) decreases performance of the inhaler as compared to the conventional actuator 2000 without a dip tube. Although MMAD was not affected by the dip tube 1906, metered dose, delivered dose, fine particle dose, and fine particle fraction were significantly reduced by adding the dip tube 1906 to the conventional actuator 2000. Despite including the dip tube 1906, the stem block 208a performed better than the conventional actuator 2000.

TABLE 6
Melatonin 152a MDIs: effect of actuation orientation

| | Melatonin Dose (μg/50 μl) | | | |
|---|---|---|---|---|
| | 100 | | | 200 |
| MDI Batch | OZ211003/RJ/A | OZ211003/RJ/B | | OZ211004/RJ/B |
| Actuator | Conventional Actuator 2000 | | Stem Block 208a | |
| Valve | Metering Valve 1908 | Metering Valve 1904 | | |
| Ethanol %(w/w) | 6 | 6 | 6 | 8 |
| Actuation | Valve Down | Valve Up | Valve Up | Valve Up |
| Orientation | | | | |
| Metered Dose | 98 ± 2 | 88 ± 7 | 103 ± 7 | 208 ± 12 |
| (μg) | | | | |
| Delivered Dose | 80 ± 3 | 73 ± 6 | 91 ± 7 | 186 ± 11 |
| (μg) | | | | |
| Fine Particle | 40 ± 2 | 31 ± 1 | 50 ± 2 | 99 ± 2 |
| Dose (μg) | | | | |
| Fine Particle | 50 ± 3 | 43 ± 1 | 55 ± 2 | 53 ± 2 |
| Fraction (%) | | | | |
| MMAD (μm) | 1.4 ± 0.1 | 1.4 ± 0.1 | 1.5 ± 0.1 | 1.6 ± 0.1 |
| n | 3 | 3 | 3 | 3 |

FIG. 23 is a graph showing the cumulative undersize (%) for melatonin delivery. The formulation comprised 100 μg/50 μl melatonin with 5% w/w ethanol with HFA 152a as the propellant. Line 2304 indicates the performance of the conventional actuator 2000 with metering valve 1908 (“valve down”), line 2308 indicates the performance of the conventional actuator 2000 with metering valve 1904 (“valve up”), and line 2312 indicates the performance of the stem block 208a (Valve up) (n=3, Mean±standard Deviation).

FIG. 24 is a graph showing the cumulative undersize (μg) for melatonin delivery. The formulation comprised 100 μg/50 μl melatonin with 5% w/w ethanol with HFA 152a as the propellant. Line 2404 indicates the performance of the conventional actuator 2000 with metering valve 1908 (“valve down”), line 2408 indicates the performance of the conventional actuator 2000 with metering valve 1904 (“valve up”), and line 2412 indicates the performance of the stem block 208a (“valve up”) (n=3, mean±standard deviation).

The metered dose determined for 100 μg/50 μl melatonin (6% w/w ethanol) when actuated with the conventional actuator 2000 was close to target or 98±2 μg. Fine particle dose <5 μm aerodynamic diameter (FPD) was 40±2 μg; and fine particle fraction <5 μm aerodynamic diameter (FPF) was observed to be 50±3%.

When the second metering valve 1908 was replaced with the first metering valve 1904 (Bespak™ with dip tube 1906) to allow for “valve up” actuation through the conventional actuator 2000 (0.30 mm), metered dose, FPD and FPF reduced to 88±7 μg, 31±4 μg and 43±1% respectively. However, when fired through the stem block 208a, the metered dose returned to 103±7 μg, and surprisingly, FPD and FPD was superior; 50±6 μg and 55±2%.

Increasing the melatonin dose to 200 μg/50 μl (8% w/w ethanol) in HFA 152a resulted in drug delivery performance being maintained if the stem block 208a was utilized; metered dose=208±12 μg, FPD=99±6 μg; FPF=53±2%.

Particle size distributions were consistent for all MDIs, as shown in FIG. 24. MMAD was 1.4-1.6 μm.

### 4.8 Melatonin, BDP & Budesonide 152a Using Stem Block 208a

Extended data is provided to include beclomethasone dipropionate (BDP) and budesonide formulated using propellant HFA 152a.

All inhalers were formulated to deliver 100 μg of the active ingredient (melatonin, BDP, or budesonide). Solubility of these formulations was previously presented in Section 4.2. Budesonide required additional ethanol (compared with melatonin and BDP) in order to obtain a formulation with a 100 μg/50 μl dose level.

Each inhaler was fitted with the metering valve 1904 (Bespak™, with dip tube 1906) and fired “valve up” through the stem block 208a and housing 104.

The results obtained in Section 4.8 are summarized in Table 7.

TABLE 7
HFA 152a MDIs: Drug Delivery from Melatonin, BDP and Budesonide MDIs

| | Dose | | |
|---|---|---|---|
| | 100 μg Melatonin | 100 μg BDP | 100 μg Budesonide |
| MDI Batch | OZ211003/RJ/B | OZ211015/DAL/A | OZ211016/DAL/A |
| Actuator | Stem Block 208a | Stem Block 208a | Stem Block 208a |
| Valve | Metering Valve 1904 | Metering Valve 1904 | Metering Valve 1904 |
| | (with dip tube 1906) | (with dip tube 1906) | (with dip tube 1906) |
| Ethanol %(w/w) | 6 | 6 | 6 |
| Actuation | Valve Up | Valve Up | Valve Up |
| Orientation | | | |
| Metered Dose | 103 ± 7 | 100 ± 5 | 93 ± 7 |
| (μg) | | | |
| Delivered Dose | 91 ± 7 | 88 ± 6 | 81 ± 7 |
| (μg) | | | |
| Fine Particle | 50 ± 2 | 49 ± 1 | 39 ± 2 |
| Dose (μg) | | | |
| Fine Particle | 55 ± 3 | 56 ± 1 | 48 ± 2 |
| Fraction (%) | | | |
| MMAD (μm) | 1.5 ± 0.1 | 1.3 ± 0.1 | 1.3 ± 0.1 |
| n | 3 | 3 | 3 |

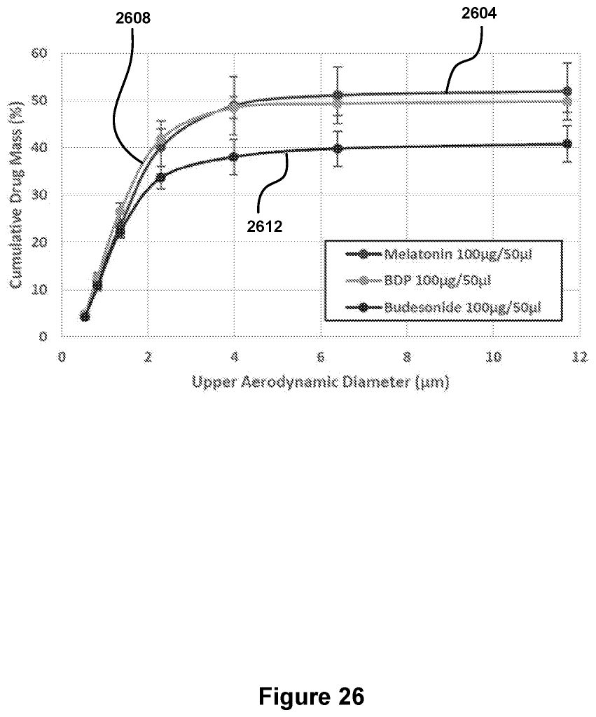

The higher content of ethanol within the budesonide formulation resulted in a lower fine particle dose (48±1 μg) compared to the melatonin (55±2 μg) and BDP (56±2 μg), however particle size distribution was consistent for all three active ingredients. The mean MMAD was 1.3-1.5 μm, as shown in Table 7 and FIG. 26.

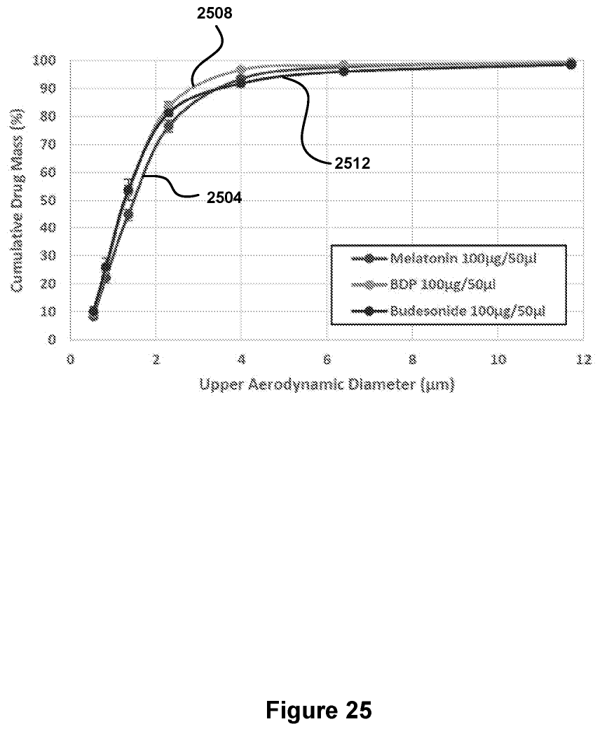

FIG. 25 is a graph showing the cumulative undersize (%) for 100 μg/50 μl melatonin in 5% w/w ethanol (indicated at 2504), 100 μg/50 μl BDP in 5% w/w ethanol (indicated at 2508), and 100 μg/50 μl budesonide in 8% w/w ethanol (indicated at 2512). The propellant was HFA 152a. The actuator was the stem block 208a (n=3, Mean±standard deviation).

FIG. 26 is graph showing the cumulative undersize (μg) for 100 μg/50 μl melatonin in 5% w/w ethanol (indicated at 2304), 100 μg/50 μl BDP in 5% w/w ethanol (indicated at 2608), and 100 μg/50 μl budesonide in 8% w/w ethanol (indicated at 2612). The propellant was HFA 152a. The actuator was the stem block 208a (n=3, Mean±standard Deviation).

### 4.9 Melatonin, BDP and Budesonide 134a Using the Stem Block

These examples extend the data set to include formulations with the propellant HFA 134a.

All formulations were formulated to deliver 100 μg of active material (melatonin, BDP or budesonide) in 12% w/w ethanol with HFA 134a as the propellant. Solubility of these formulations was presented in Section 4.3.

The results were evaluated for various combinations of actuator, valve, and actuation orientation.

Study 4a: Metering valve 1908 (Aptar™, without dip tube) fired “valve up” and “valve down” with the conventional actuator 2000, compared to metering valve 1904 (Bespak™ with dip tube 1906) fired “valve up” with either a conventional actuator 2000 or the stem block 208a.

Study 4b: Metering valve 1908 (Aptar™, without dip tube) fired “valve up” through the conventional actuator 2000.

Study 4c: Metering valve 1904 (Bespak™ with dip tube 1906) and fired “valve up” through the stem block 208a and housing 104.

In Studies 4a and 4b, where necessary, the respective inhaler is rotated into the “filling” orientation after actuation/dosing, as shown in FIG. 20C, but prior to releasing the valve to its rest position. This process allows the evaluation of drug delivery performance from the conventional actuator 2000 without a dip tube that is fired “valve up” (i.e., non-conventionally).

### 4.10 Melatonin Delivery for Conventional Actuators and Stem Block

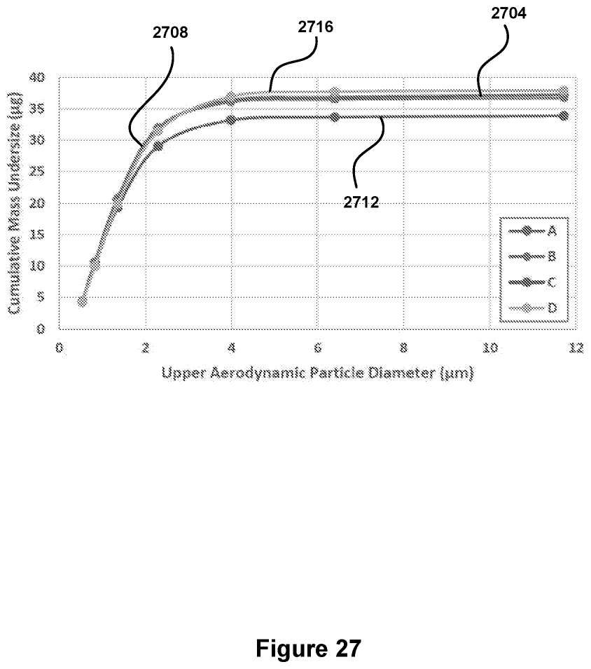

Table 8 and FIG. 27 presents melatonin delivery data with HFA 134a for (A) the conventional actuator 2000 fired “valve down”; (B) the conventional actuator 2000 fired “valve up”; (C) the conventional actuator 2000 with dip tube 1906 fired “valve up”; and (D) the stem block 208a with dip tube 1906 fired “valve up”.

Melatonin delivered in the “valve up” orientation using the stem block 208a is observed to be at least as efficient as delivery from the conventional actuator 2000, as shown in Table 8. Table 8 shows that the FPD from A, B, C and D was 37±1, 36±3, 33±2, and 37±1 respectively.

Particle size distributions for all MDIs were similar and the MMAD for all MDIs was 1.2-1.3 μm.

Table 8 shows that the dip tube 1906 added to the conventional actuator 2000 (C) decreases performance of the inhaler as compared to a conventional actuator 2000 without the dip tube 1906 either fired valve up (B) or valve down (A). Although MMAD was not affected by the dip tube 1906, metered dose, delivered dose, fine particle dose, and fine particle fraction were significantly reduced by adding the dip tube 1906 to the conventional actuator. Despite having the dip tube 1906, the stem block 208a performed just as well or better than the conventional actuator.

TABLE 8
HFA 134a: Melatonin delivery from valves fired
upright & inverted (each orientation ± dip tube)

| | FIG. 27 Key: | | | |
|---|---|---|---|---|
| | A (line 2704) | B (line 2708) | C (line 2812) | D (line 2816) |
| MDI Batch | OZ211025/DAL/A | OZ211025/DAL/A | OZ211025/DAL/B | OZ211025/DAL/A |
| Actuator | Conventional | Conventional | Conventional | Stem Block 208a |
| | Actuator 2000 | Actuator 2000 | Actuator 2000 | |
| Valve | Metering Valve | Metering Valve | Metering Valve | Metering Valve |
| | 1908 (without dip | 1908 (without dip | 1904 (with dip | 1904 (with dip |
| | tube) | tube) | tube 1906) | tube 1906) |
| Ethanol | 12 | 12 | 12 | 12 |
| %(w/w) | | | | |
| Actuation | Valve Down | Valve Up | Valve Up | Valve Up |
| Orientation | | | | |
| Metered | 97 ± 2 | 94 ± 2 | 91 ± 3 | 94 ± 2 |
| Dose (μg) | | | | |
| Delivered | 84 ± 1 | 79 ± 3 | 77 ± 3 | 86 ± 1 |
| Dose (μg) | | | | |
| Fine | 37 ± 1 | 36 ±3 | 33 ± 3 | 37 ± 1 |
| Particle | | | | |
| Dose (μg) | | | | |
| Fine | 44 ± 1 | 46 ± 3 | 43 ± 1 | 44 ± 1 |
| Particle | | | | |
| Fraction | | | | |
| (%) | | | | |
| MMAD | 1.3 ± 0.1 | 1.2 ± 0.1 | 1.2 ± 0.1 | 1.3 ± 0.1 |
| (μm) | | | | |
| n | 3 | 3 | 3 | 3 |

FIG. 27 is a graph showing the cumulative mass undersize (μg) for A (indicated at 2704), B at (indicated at 2708), C (indicated at 2712), and D (indicated at 2716).

### 4.11 Conventional Actuator Versus Stem Block (Melatonin, BDP & Budesonide)

The data presented in Table 9 and Table 10 further extends the data set to include alternative molecules; Beclomethasone Dipropionate (BDP) and Budesonide formulated using the propellant HFA 134a. Solubility for these formulations was confirmed in Section 4.3.

All formulations contained 12% w/w ethanol with HFA 134a as the propellant. The MDIs were fired “valve up”. Table 9 shows the results for the Presspart™ 0.30 mm conventional actuator 2000 and Table 10 shows the results for the stem block 208a.

Consistent drug delivery data was observed for the three molecules evaluated (melatonin, BDP and budesonide). Mean metered dose values were consistent between both actuator types (94-97 μg). However, the mean delivered dose was lower for the conventional actuator 2000 (79-84 μg) than for the stem block 208a and housing 104 (86-87 μg). The mean fine particle dose observed was 36-37 μg for the conventional actuator 2000 and 37-39 μg when delivered using the stem block 208a.

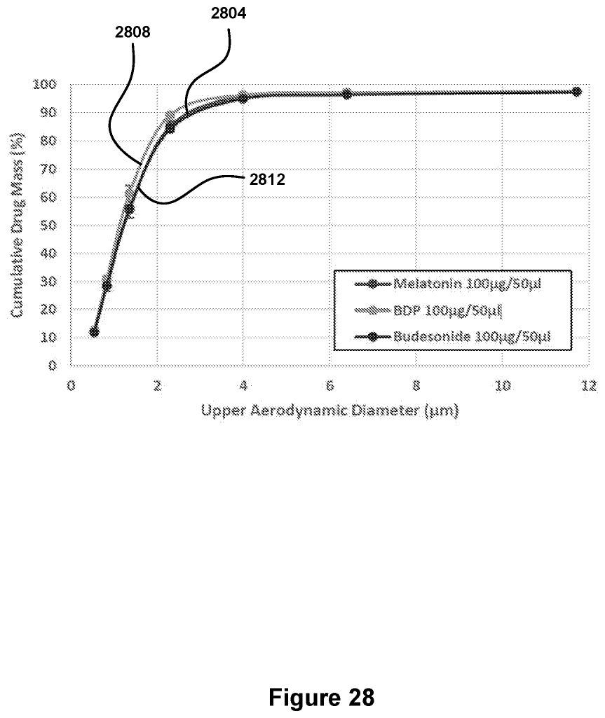

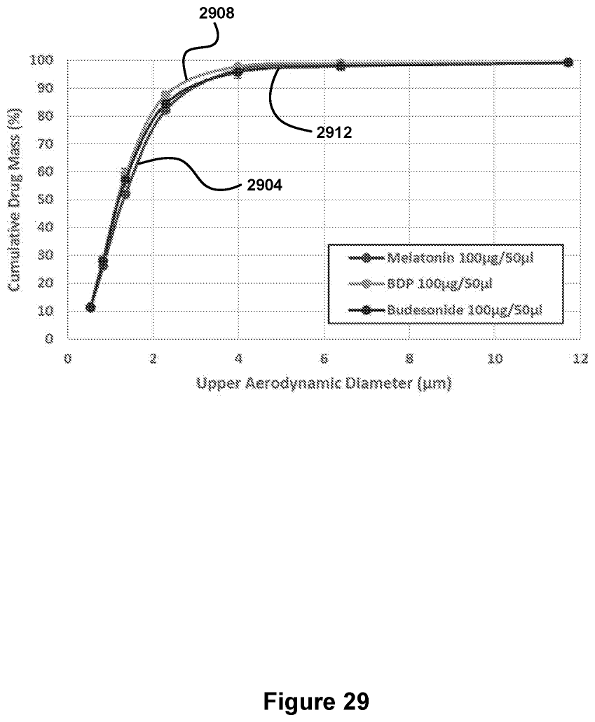

Particle size distributions were consistent (MMAD=1.1-1.3 μm) for all measurements (see FIG. 28 and FIG. 29).

TABLE 9
Presspart ™ Conventional Actuator (Melatonin, BDP and Budesonide)

| | Dose | | |
|---|---|---|---|
| | 100 μg Melatonin | 100 μg BDP | 100 μg Budesonide |
| MDI Batch | OZ211025/DAL/A | OZ211027/DAL/A | OZ211016/DAL/A |
| Actuator | Conventional Actuator 2000 (Presspart ™, 0.3 mm) | | |
| Valve | Metering Valve 1908 (Aptar ™, without dip tube) | | |
| Ethanol | 12 | 12 | 12 |
| %(w/w) | | | |
| Actuation | Valve Down | Valve Up | Valve Up |
| Orientation | | | |
| Metered Dose | 94 ± 2 | 95 ± 3 | 97 ± 1 |
| (μg) | | | |
| Delivered | 79 ± 3 | 79 ± 3 | 84 ± 2 |
| Dose (μg) | | | |
| Fine Particle | 36 ± 3 | 36 ± 4 | 37 ± 1 |
| Dose (μg) | | | |
| Fine Particle | 46 ± 3 | 45 ± 1 | 44 ±2 |
| Fraction (%) | | | |
| MMAD (μm) | 1.2 ± 0.1 | 1.1 ± 0.1 | 1.2 ± 0.1 |
| n | 3 | 3 | 3 |

FIG. 28 is a graph showing the cumulative undersize (μg) for melatonin (indicated at 2804), BDP at (indicated at 2808) and budesonide at (indicated at 2812). The concentration of the active ingredient in each formulation was 100 μg/50 μl. The concentration of ethanol was 12% w/w. The propellant was HFA 134a. The actuator was the Conventional Actuator 2000 (Presspart™, 0.30 mm) (n=3, Mean±standard Deviation).

TABLE 10
Stem Block (Melatonin, BDP and Budesonide)

| | Dose | | |
|---|---|---|---|
| | 100 μg Melatonin | 100 μg BDP | 100 μg Budesonide |
| MDI Batch | OZ211025/DAL/B | OZ211027/DAL/B | OZ211029/DAL/B |
| Actuator | Stem Block 208a | | |
| Valve | Metering Valve 1904 (with the dip tube 1906) | | |
| Ethanol | 12 | 12 | 12 |
| %(w/w) | | | |
| Actuation | Valve Up | Valve Up | Valve Up |
| Orientation | | | |
| Metered Dose | 94 ± 2 | 95 ± 1 | 96 ± 2 |
| (μg) | | | |
| Delivered | 86 ± 1 | 86 ±2 | 87 ± 2 |
| Dose (μg) | | | |
| Fine Particle | 37 ± 1 | 39 ± 1 | 38 ± 3 |
| Dose (μg) | | | |
| Fine Particle | 44 ± 1 | 46 ± 2 | 44 ± 2 |
| Fraction (%) | | | |
| MMAD (μm) | 1.3 ± 0.1 | 1.2 ± 0.1 | 1.2 ± 0.1 |
| n | 3 | 3 | 3 |

FIG. 29 is a graph showing drug delivery using the stem block 208a. The cumulative undersize (μg) for melatonin is indicated at line 2904, BDP is indicated at line 2908, and budesonide is indicated at line 2912. The concentration of the active ingredient in each formulation was 100 μg/50 μl. The concentration of ethanol was 12% w/w. The propellant was HFA 134a (n=3, Mean±standard Deviation).

### 4.12 Performance of Melatonin 50 μg/50 μl (5% w/w Ethanol)

Section 4.12 extends the data set to include MDIs 50 μg/50 μl Melatonin formulated with “low” 5% w/w ethanol content using the propellant HFA 134a. The solubility for this formulation is confirmed in Section 4.3.

Section 4.12 compares the drug delivery for the conventional actuator 2000 to the metering valve 1904 fitted with the dip tube 1906 valve fired “valve up” through the stem block 208a.

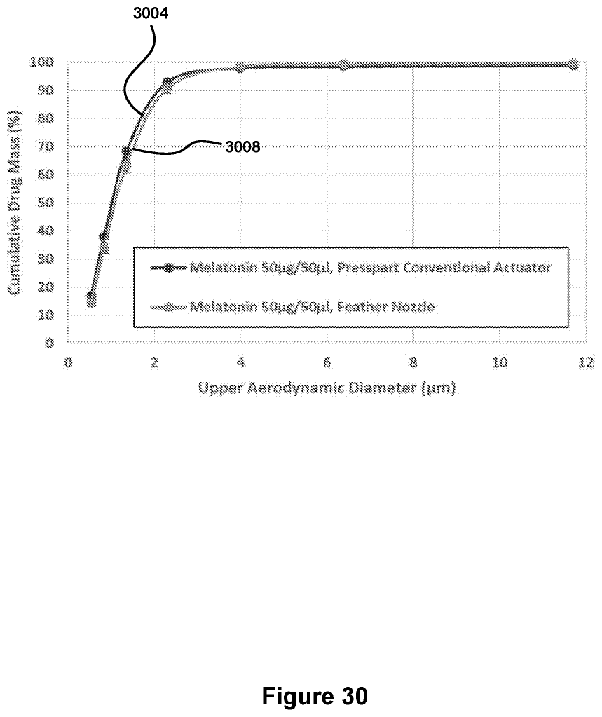

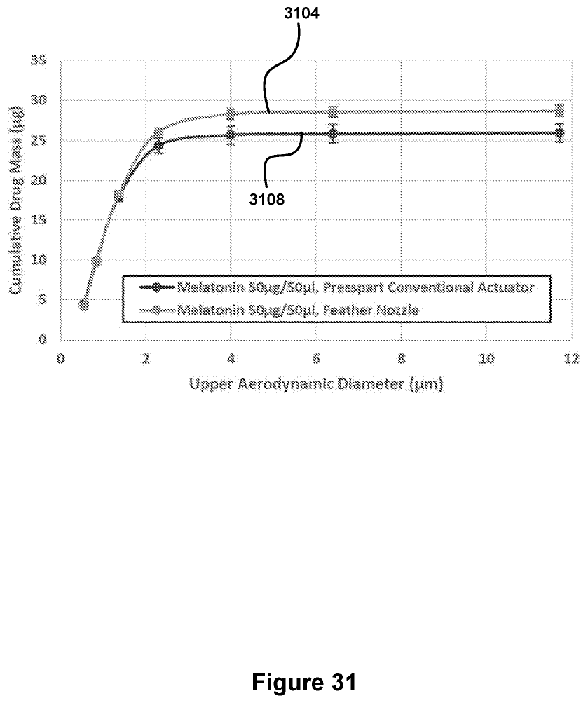

Results obtained in Section 4.12 are summarized in Table 11, FIG. 30 and FIG. 31.

As shown in FIG. 30, the particle size distributions (%) are similar for the conventional actuator 2000 and the stem block 208a (MMAD=1.0-1.1 μm).

A fine particle fraction of 62±4% was obtained from the conventional actuator 2000 compared with 67±1% from the stem block 208a.

The FPD observed for the stem block 208a (FPD=28±1 μg) was at least as good as when using the conventional actuator 2000 (FPD=26±1 μg).

TABLE 11
HFA 134a MDIs: Drug Delivery from Melatonin with 5% w/w Ethanol

Dose

| | 50 μg Melatonin | 50 μg Melatonin |
|---|---|---|
| MDI Batch | OZ211103/DAL/A | OZ211103/DAL/B |
| Actuator | Conventional Actuator | Stem Block 208a |
| | 2000 | |
| Valve | Metering Valve 1908 | Metering Valve 1904 (with dip |
| | (without dip tube) | tube 1906) |
| Ethanol %(w/w) | 5 | 5 |
| Actuation | Valve Down | Valve Up |
| Orientation | | |
| Metered Dose (μg) | 50 ± 2 | 48 ± 1 |
| Delivered Dose (μg) | 42 ± 1 | 43 ± 1 |
| Fine Particle Dose | 26 ± 1 | 28 ± 1 |
| (μg) | | |
| Fine Particle | 62 ± 4 | 67 ± 1 |
| Fraction (%) | | |
| MMAD (μm) | 1.0 ± 0.1 | 1.1 ± 0.1 |
| n | 3 | 3 |

FIG. 30 is a graph showing the cumulative undersize (%) for 50 μg/μl melatonin in 5% w/w ethanol delivered using HFA 134a as the propellant. Delivery with the conventional actuator 2000 (“valve down”) is indicated at line 3004 and delivery with the stem block 208a (“valve up”) is indicated at line 3008 (n=3, Mean±standard Deviation).

FIG. 31 is a graph showing the cumulative undersize (μg) for 50 μg/μl melatonin in 5% w/w ethanol delivered using HFA 134a as the propellant. Delivery with the conventional actuator 2000 (“valve down”) is indicated at line 3104 and delivery with the stem block 208a (“valve up”) is indicated at line 3108 (n=3, Mean±standard Deviation).

### 4.13 Stability of Melatonin in Formulation

MDI solution formulations containing 100 μg/50 μl melatonin in 6% w/w ethanol using HFA 152a as the propellant were evaluated for stability in Presspart™ 14 ml canisters: plain aluminum or plasma surface treated. Each canister type was packaged with metering valve 1908 (Aptar™, 50 μl) (MDI batches OZ211003/RJ/A and OZ211003/RJ/C) and metering valves 1904 (Bespak™ 50 μl, with dip tube 1906) (MDI batches OZ211003/RJ/B and OZ211003/RJ/D).

Table 12 presents melatonin residual following 0-month, 1-month and 3-month storage at 40° C. and 75% relative humidity for MDIs stored either “valve up” or “valve down”.

TABLE 12
Drug residual of melatonin 100 μg/50 μL in 6% w/w ethanol, HFA 152a MDIs.

| | | | | Residual Melatonin (MDI storage orientation) | | | | | | | |
|---|---|---|---|---|---|---|---|---|---|---|---|
| | | Initial Melatonin | | t = 1 month @ 40° C. | | | | | | | |
| | | t = 0 | | | | | | t = 3 month @ 40° C. | | | |
| Presspart ™ | | Upright | | Upright | | Inverted | | | % | | % |
| 14 ml | Valve Type | | mean | | % of | | % of | | of | | of |
| Canister | (50 μg) | μg | (mg) | μg | t = 0 | μg | t = 0 | μg | t = 0 | μg | t = 0 |
| Plasma | Metering | 15430 | 15.6 | 15578 | 100 | 15024 | 96 | 15461 | 100 | 14661 | 94 |
| coated | Valve 1908 | 15517 | | 15444 | | 14980 | | 15469 | | 14791 | |
| | | 15810 | | — | | — | | — | | — | |
| Plasma | Metering | 15417 | 15.5 | 14944 | 98 | 14872 | 97 | 14805 | 98 | 14634 | 94 |
| Coated | Valve 1904 | 15682 | | 15211 | | 14984 | | 14789 | | 14510 | |
| | (with dip tube | 15292 | | — | | — | | — | | — | |
| | 1906) | | | | | | | | | | |
| Plain | Metering | 16200 | 15.8 | 15403 | 100 | 15171 | 98 | 15600 | 100 | 14920 | 96 |
| Aluminum | Valve 1908 | 15676 | | 15581 | | 15131 | | 15422 | | 14914 | |
| | | 15379 | | — | | — | | — | | — | |
| Plain | Metering | 15408 | 15.6 | 14959 | 97 | 15162 | 97 | 14721 | 98 | 14652 | 93 |
| Aluminum | Valve 1904 | 15450 | | 15331 | | 14967 | | 14847 | | 14318 | |
| | (with dip tube | 15897 | | — | | — | | — | | — | |
| | 1906) | | | | | | | | | | |

The initial time point residual data was 97.5±1.7% of the target formulation.

Residual melatonin at 1-month was 96-98% for all packaging and storage conditions.

For inhalers stored “valve up”, residual melatonin at 3-month for both canister types were 100% when packaged with metering valve 1908 (Aptar™) and 98% when packaged with metering valve 1904 (Bespak™ with dip tube 1906). For MDIs stored “valve down”, residual melatonin at 3-month ranged from 93% to 96%.

### 4.14 Stability of Melatonin Formulations

The stability of a melatonin formulation including HFA-152a was tested over a 3-month period.

The formulation comprised 100 μg/50 μL melatonin and 6% w/w ethanol in HFA 152a. The results of the trial are displayed in Table 13

TABLE 13
Melatonin (100 μg) Chemical Stability at 0-months, 1-month, 3-month

| | | Initial Melatonin | | Residual Melatonin (MDI storage orientation) | | | | | | | |
|---|---|---|---|---|---|---|---|---|---|---|---|
| | | t = 0 | | t = 1 month @ 40° C. | | | | | | | |
| Presspart ™ | | Upright | | Upright | | Inverted | | t = 3 month @ 40° C. | | | |
| 14 ml | Valve Type | | mean | | % of | | % of | | % of | | % of |
| Canister | (50 μg) | μg | (mg) | μg | t = 0 | μg | t = 0 | μg | t = 0 | μg | t = 0 |
| Plasma | Metering Valve | 15430 | 15.6 | 15578 | 100 | 15024 | 96 | 15461 | 100 | 14661 | 94 |
| coated | 1908 | 15517 | | 15444 | | 14980 | | 15469 | | 14791 | |
| | | 15810 | | — | | — | | — | | — | |
| Plasma | Metering Valve | 15417 | 15.5 | 14944 | 98 | 14872 | 97 | 14805 | 98 | 14634 | 94 |
| Coated | 1904 (with dip | 15682 | | 15211 | | 14984 | | 14789 | | 14510 | |
| | tube 1906) | 15292 | | — | | — | | — | | — | |
| Plain | Metering Valve | 16200 | 15.8 | 15403 | 100 | 15171 | 98 | 15600 | 100 | 14920 | 96 |
| Aluminum | 1908 | 15676 | | 15581 | | 15131 | | 15422 | | 14914 | |
| | | 15379 | | — | | — | | — | | — | |
| Plain | Metering Valve | 15408 | 15.6 | 14959 | 97 | 15162 | 97 | 14721 | 98 | 14652 | 93 |
| Aluminum | 1904 (with dip | 15450 | | 15331 | | 14967 | | 14847 | | 14318 | |
| | tube 1906) | 15897 | | — | | — | | — | | — | |

### 4.15 Drug Delivery of Melatonin Formulations

Further tests were conducted to evaluate the drug delivery of two melatonin formulations: a 100 μg/50 μL formulation and a 200 μg/μL formulation. The two formulations are described in Table 14 and Table 15.

TABLE 14
100 μg/50 μL melatonin formulation

| 100 μg/ | | Mass per | Dose per |
|---|---|---|---|
| 50 μl | % (w/w) | canister (mg) | 50 μl (mg) |
| Melatonin | 0.224 | 15.990 | 0.100 |
| Ethanol | 5.000 | 357.400 | 2.236 |
| HFA 152a | 94.776 | 6774.610 | 42.376 |
| Total | 100.000 | 7148.000 | 44.711 |

TABLE 15
200 μg/50 μL melatonin formulation

| 200 μg/ | | Mass per | Dose per |
|---|---|---|---|
| 50 μl | % (w/w) | canister (mg) | 50 μl (mg) |
| Melatonin | 0.449 | 32.095 | 0.200 |
| Ethanol | 8.000 | 571.840 | 3.564 |
| HFA 152a | 91.551 | 6544.065 | 40.786 |
| Total | 100.00 | 7148.000 | 44.550 |

20 Presspart™ cannisters of the 100 μg/50 μL melatonin formulation were prepared with 0.23 mm actuators. 20 Presspart™ cannisters of the 200 μg/50 μL melatonin formulation were prepared with 0.23 mm actuators. Each cannister contained 160 doses (120 target doses and 40 overage doses).

The results of the evaluation are shown below in Table 16.

TABLE 16

| | 100 μg | 200 μg |
|---|---|---|
| Metered Dose [μg] | 98 ± 1 | 195 ± 3 |
| Delivered Dose [μg] | 85 ± 1 | 171 ± 1 |
| Fine Particle Dose [μg] | 56 ± 4 | 95 ± 7 |
| Fine Particle Fraction [%] | 67 ± 5 | 56 ± 4 |
| MMAD [μm] | 1.5 ± 0.1 | 1.7 ± 0.1 |

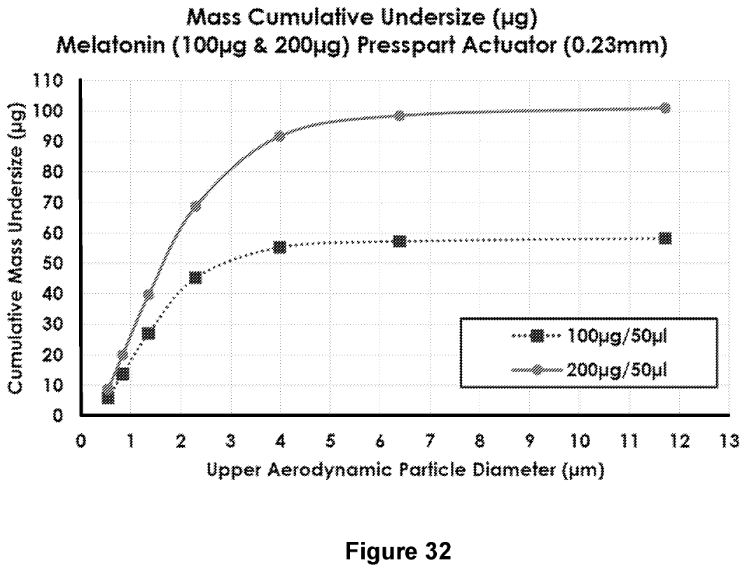

The mass cumulative undersize (μg) for the two formulations is shown in FIG. 32.

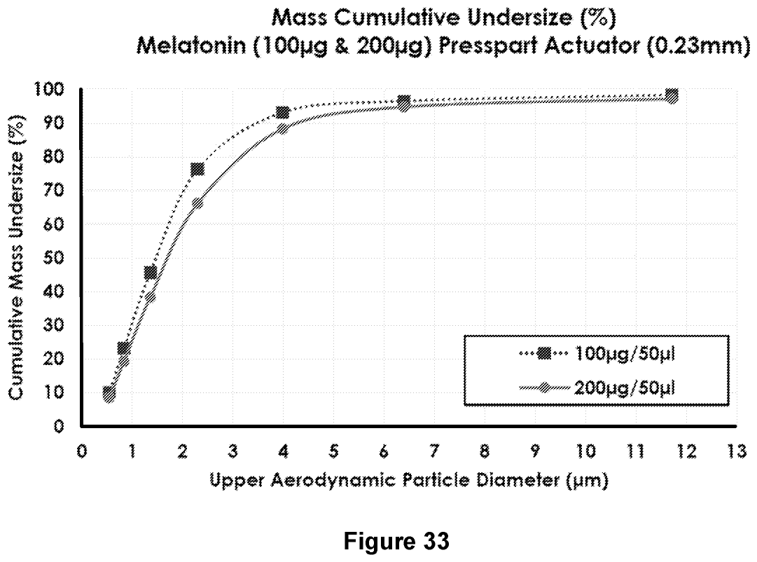

The mass cumulative undersize (%) for the two formulations is shown below in FIG. 33.

### Example 2: Nicotine

### 5.1 Stability of Nicotine in Formulation

The stability of two therapeutic formulations comprising nicotine were tested for stability over a 4.5-month period.

The first therapeutic formulation comprises 0.1676% (w/w) nicotine, 5% (w/w) ethanol, 0.1% (w/w) glycerol, 0.04% (w/w) saccharin, and 94.6924% HFA 134a. The first therapeutic formulation is described in Table 17 below.

TABLE 17
Composition of First Formulation

| | | Mass per | Dose per |
|---|---|---|---|
| | % (w/w) | canister (mg) | 50 μl (mg) |
| Nicotine (L- | 0.1676 | 24 | 0.1000 (100 μg) |
| Nicotine) CAS 55- | | | |
| 11-5 | | | |
| Ethanol | 5 | 715.7 | 2.9822 |
| Glycerol | 0.1 | 14.3 | 0.0596 |
| Saccharin | 0.04 | 5.7258 | 0.0239 |
| HFA 134a | 94.6924 | 13566.7 | 56.4767 |
| Total | 100 | 14326.43 | 59.6423 |

The second therapeutic formulation comprises 0.2240% (w/w) nicotine, 5% (w/w) ethanol, 0.1% (w/w) glycerol, 0.04% (w/w) saccharin, and 94.6924% HFA 152a. The second therapeutic formulation is described in Table 18 below.

TABLE 18
Composition of Second Formulation

| | | Mass per | Dose per |
|---|---|---|---|
| | % (w/w) | canister (mg) | 50 μl (mg) |
| Nicotine (L- | 0.2240 | 32 | 0.1000 (100 μg) |
| Nicotine) CAS 55- | | | |
| 11-5 | | | |
| Ethanol | 5 | 715.7 | 2.236 |
| Glycerol | 0.1 | 14.3 | 0.0447 |
| Saccharin | 0.04 | 5.7258 | 0.0179 |
| HFA 134a | 94.6924 | 13566.7 | 24.3259 |
| Total | 100 | 14326.43 | 44.7250 |

A plurality of metered dose inhalers (MDIs) were filled with either the first or second therapeutic formulation. MDIs were stored either upright (Up) or inverted (in).

Each MDI comprised a valve and a canister. Three different valves were tested: Aptar 50 μl, for inverted use (Batch: BE-ECHPI-ASS-2021-66-V1), Bespak 50 μl for upright use (dip tube) (Batch: OZ210406_A), and Bespak valve for glass bottle. Three different canisters were tested: Presspart i-Plasma 14 ml, Presspart i-Plasma 19 ml and St Gobain Glass.

The MDIs were stored for 4.5 months at 5° C., 25° C., or 40° C. Subsequently, the MDIs were evaluated to determine the storage effects on drug delivery, can content, and valve compatibility. The results are shown below in Table 19.

TABLE 19
Stability of Nicotine HFA143a formulation and Nicotine-HFA 152a formulation

| | | | Temp | Valve Up | Nicotine | |
|---|---|---|---|---|---|---|
| Propellant | Valve | Canister | (% RH) | or Down? | μg | % of 5° C. |
| HFA 134a | Metering Valve 1908 | Presspart ™ | 5° C. | Up | 24166.562 | 100 |
| | | Plasma | | | 24126.474 | |
| | | Coated 14 ml | 25° C. | Up | 23922.695 | 98 |
| | | C128 | | | 23527.533 | |
| | | | 40° C. (75% RH) | Up | 22809.747 | 97 |
| | | | | | 23785.869 | |
| | | | 40° C. (75% RH) | Down | 23663.281 | 98 |
| | | | | | 23663.281 | |
| HFA 134a | Metering valve 1904 | | 5° C. | Up | 23891.217 | 100 |
| | (with dip tube 1906) | | | | 23628.856 | |
| | | | 40° C. (75% RH) | Up | 22360.125 | 96 |
| | | | | | 23044.004 | |
| | | | 40° C. (75% RH) | Down | 23102.878 | 97 |
| | | | | | 23079.598 | |
| HFA 152a | Metering Valve 1908 | | 5° C. | Up | 31626.434 | 100 |
| | | | | | 30855.835 | |
| | | | 25° C. | Up | 31592.559 | 100 |
| | | | | | 30786.35 | |
| | | | 40° C. (75% RH) | Up | 30982.764 | 98 |
| | | | | | 30318.689 | |
| | | | 40° C. (75% RH) | Down | 30939.368 | 98 |
| | | | | | 30153.5 | |
| HFA 152a | Metering valve 1904 | | 40° C. (75% RH) | | 30395.997 | 97 |
| | (with dip tube 1906) | | | | 30048.645 | |

The therapeutic formulation may be selected for delivery in a sublingual spray device, as described herein.

### 5.2 Solubility of Nicotine in Formulation

The solubility of nicotine and caffeine was tested in propellant gases. The results are summarized in Table 20:

TABLE 20
Solubility of the caffeine and nicotine base in different propellant systems

| | Mean Drug Solubility (μg/ml ± standard deviation) | | | | | |
|---|---|---|---|---|---|---|
| | | | | | 50:50 HFA | 50:50 HFA |
| Active | HFA | HFA | HFA | HFO | 134a/HFA | 134a/HFO |
| Agent | 134a | 227ea | 152a | 1234ze | 152a | 1234ze |
| Caffeine | 389 ± 12 | 285 ± 17 | 1326 ± 27 | 345 ± 28 | 1418 ± 39 | 842 ± 25 |
| Nicotine (L- | 38.5 ± 1.6 | 19.8 ± 4.9 | 50.8 ± 2.9 | 41.5 ± 0.8 | 85.8 ± 3.2 | 62.3 ± 4.4 |
| Nicotine) - | | | | | | |
| CAS 55-11-5 | | | | | | |

Four propellants were tested: HFA 134a, HFA 227ea, HFA 152a, and 1,3,3,3-Tetrafluoropropene (HFO 1234ze).

The solubility for nicotine in HFA 152a is 50.8±2.9 μg/ml. This is equivalent to 0.56% by mass.

A surprising result is that the 50:50 mixtures of propellants demonstrated higher solubility than either propellant alone. This was true for both active agents: nicotine and caffeine.

The solubility of nicotine was further investigated in an ethanol solution. The two samples tested below comprise HFA 134a and 0% (w/w) or 14.8% (w/w) ethanol. Nicotine was gradually added (1.5 mg per 50 μl) to find the upper limit of solubility.

The results are shown in Table 21:

TABLE 21
Nicotine solubility in ethanol

| | | Nicotine Detected in Solution | | Detected/ |
|---|---|---|---|---|
| Ethanol | Nicotine | (by HPLC) | | Added |
| %(w/w) | Added %(w/w) | % (w/w) | μg per 50 μl | *100% |
| 0 | 0.169 ± 0.009 | 0.168 ± 0.026 | 104 ± 15 | 99 ± 11 |
| 14.8 | 0.703 | 0.760 ± 0.013 | 421 ± 10 | 108 ± 2 |

As shown in Table 21, solubility of at least 10 μg/ml is attainable in HFA 134a. the solubility of nicotine in HFA 134a is greatly improved with the addition of ethanol.

### 5.3 Drug Delivery of Nicotine Formulation

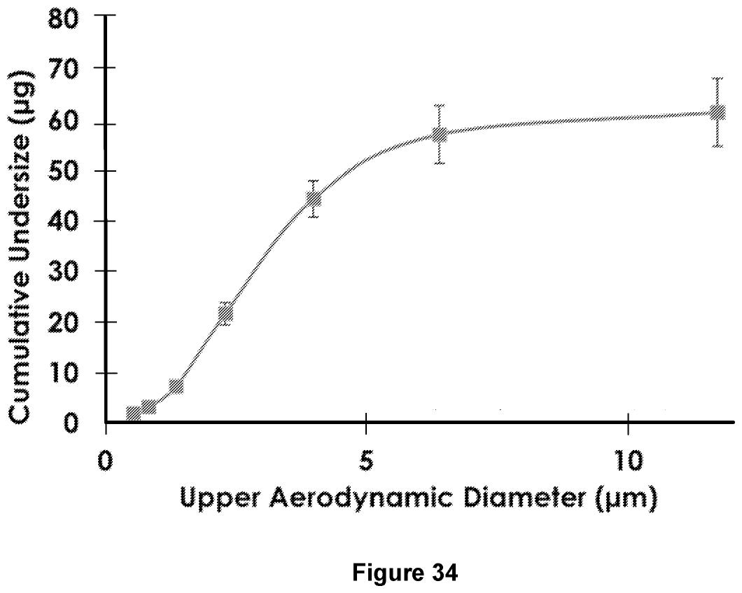

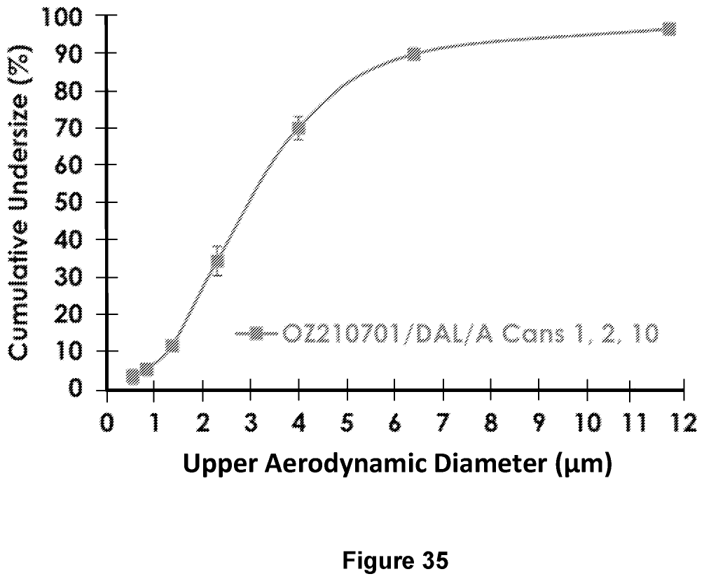

Cascade impactor testing was conducted on the first formulation described in Table 17. The conventional actuator had a 0.30 mm orifice and the valve was metering valve 1908 (50 μl). The results are shown in FIGS. 34 and 35 and Table 22 below:

TABLE 22

| NGI | Nicotine 100 μg/50 μl |
|---|---|
| Batch | OZ210701/DAL/A |
| Cans | 1, 2, 10 |
| NGI ID# | 230, 231, 232 |
| Metered Dose (μg) | 105.4 ± 9.5 |
| Delivered Dose (μg) | 99.4 ± 9.8 |
| Fine Particle Dose (μg) | 51.6 ± 4.7 |
| Fine Particle Fraction (%) | 51.9 ± 1.3 |
| MMAD (μm) | 2.9 ± 0.2 |
| GSD | 1.8 ± 0.0 |
| Shot Weight (mg) | 60.6 ± 0.9 |
| Vapor Trap (μg) | 1.4 ± 0.8 |
| Vapor Trap (%) | 1.3 ± 0.8 |

### 6 Example 3: CBD

### 6.1 Stability of CBD in Formulation

The stability of a cannabidiol (CBD) formulation in HFA 134a was evaluated over a 6-month period.

The formulation consisted of 3.57% (w/w) CBD, 15% (w/w) ethanol, and HFA 134a. The formulation was packaged in Presspart™ Plasma 14 mL canisters with metering valves 1908 (Aptar™). The canisters were stored in the valve-up orientation for 6 months at 5° C., 25° C., or 40° C. The residual formulation was measured at 1 month and 6 months. The results are shown below in Table 23.

TABLE 23

| | Residual (% of 5° C. data*) | | | | | | |
|---|---|---|---|---|---|---|---|
| | | | | 6 | | | |
| | | | | month | | | |
| | | | | (n = 4) | | | |
| | | | | Valve | Manufacture Conditions | | |
| | 1 month (n = 3) Valve Up | | | Up | O₂ | RH | T |
| Batch | 5° C. | 25° C. | 40° C. | 25° C. | (%) | (%) | (° C.) |
| OZ210527/ | 100.00 ± | 100.3 ± | 98.5 ± | 93.3 ± | 2.5 ± | 17.8 ± | 24.0 ± |
| DAL/G1 | 2.6 | 1.7 | 0.2 | 2.6 | 0.7% | 1.7% | 0.1° C. |
| OZ210527/ | 100.0 ± | 100.2 ± | 98.1 ± | 93.5 ± | 0.8 ± | 5.8 ± | 23.3 ± |
| DAL/H1 | 1.1 | 1.3 | 0.3 | 1.0 | 0.2% | 1.6% | 0.3° C. |

The results show that CBD-HFA formulations are stable at 5° C., 25° C. and 40° C.

### 7 Conclusions

Metered dose inhalers (MDIs) are conventionally atomized “valve down” using valves that do not have dip tubes. This report demonstrates that the stem block of the present specification allows for the actuation of an MDI with a dip tube in the “valve up” orientation without loss of drug delivery performance. Furthermore, drug delivery has been demonstrated for three different molecules (melatonin, BDP and budesonide) to be at least equivalent to that of MDIs using conventional MDI hardware and conventional actuators in the conventional “valve down” orientation. Having regards to metered dose, delivered dose, fine particle dose/fraction less than 5 μm aerodynamic diameter and particle size distribution of the therapeutic dose; drug delivery using the stem block is at least equivalent (and superior in some examples) to the conventional actuators.

Example 1 demonstrates the delivery of a formulation comprising 100 μg/50 μl melatonin, 6% w/w ethanol and HFA 152a. When actuated conventionally in the “valve down” orientation using a conventional actuator 2000 having a 0.30 mm orifice, the fine particle dose (FPD) observed was 40±2 μg. When the same formulation was fired “valve up” with a 50 μl dip tube valve and the same conventional actuator, the FPD dropped to 31±4 μg. Replacing the conventional actuator with the stem block 208 increased the FPD to 50±6 μg.

In the above-described examples, the performance of the stem block 208 is demonstrated using formulations including the propellant HFA 134a in addition to formulations including the environmentally sensitive propellant HFA 152a. In particular, the disclosed formulation comprising a low global warming potential HFA 152a is of considerable interest due to the recent changes in quotas that regulate the supply of F-Gases such as HFA 134a. This report demonstrates that formulations comprising HFA 152a are suitable for delivering a therapeutically effective dose of melatonin. Such formulations may be suitable for treating conditions such as insomnia, narcolepsy and other sleep disorders. Administering therapeutic agents by inhalation may induce a more rapid physiological response than administration by ingestion. It is anticipated that, active agents such as melatonin, caffeine, nicotine, and cannabidiol will enter the systemic blood stream within about 15 seconds of inhalation, thus avoiding first pass metabolism by the kidneys and liver as would be encountered by an orally administered drug.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

1. A therapeutic formulation for sublingual or pulmonary delivery of an active agent, the therapeutic formulation comprising:
a therapeutically effective amount of the active agent;
a propellant; and
a co-solvent.

2. The therapeutic formulation of claim 1 wherein the active agent includes nicotine.

3. The therapeutic formulation of claim 2 wherein the nicotine comprises between 0.22 to 3% of the formulation by weight.

4. (canceled)

5. The therapeutic formulation of claim 2 wherein the propellant includes hydrofluoroalkane 134a.

6. The therapeutic formulation of claim 2 wherein the propellant includes a mixture of hydrofluoroalkane 152a and hydrofluoroalkane 134a.

7. The therapeutic formulation of claim 2 wherein the propellant includes hydrofluoroalkane 152a.

8. The therapeutic formulation of claim 7 wherein the propellant comprises between 80% and 98% of the formulation by weight.

9. (canceled)

10. The therapeutic formulation of claim 2 wherein the co-solvent includes ethanol.

11. The therapeutic formulation of claim 10 wherein the co-solvent comprises between 0% and 15% of the formulation by weight.

12. The therapeutic formulation of claim 2 further comprising an anti-nucleating agent.

13. The therapeutic formulation of claim 12 wherein the anti-nucleating agent includes propylene glycol.

14. The therapeutic formulation of claim 2 further comprising a flavouring agent.

15. The therapeutic formulation of claim 14 wherein the flavouring agent comprises between 0 to 3% of the formulation by weight.

16. The therapeutic formulation of claim 15 wherein the flavouring agent includes menthol.

17. The therapeutic formulation of claim 16 wherein the menthol comprises 0.77±0.08% by weight of the formulation.

18. The therapeutic formulation of claim 1, comprising
hydrofluoroalkane 152a comprising 95.92±9% by weight of the formulation;
ethanol comprising 1.70±0.2% by weight of the formulation;
water comprising 0.41±0.04% by weight of the formulation;
nicotine comprising 0.74±0.07% by weight of the formulation; and
the formulation further comprising menthol comprising 0.77±0.08% by weight of the formulation; and
a flavouring agent comprising 0.46±0.05% by weight of the formulation.

19. The therapeutic formulation comprising:
hydrofluoroalkane 152a comprising 94.69±0.5% by weight of the formulation;
ethanol comprising 5±1% by weight of the formulation;
glycerol comprising 0.1±0.1% by weight of the formulation;
nicotine comprising 0.22±0.3% by weight of the formulation; and
saccharin comprising 0.04±0.05% by weight of the formulation.

20. The therapeutic formulation of claim 1, comprising:
hydrofluoroalkane 152a comprising 81.5±3% by weight of the formulation;
ethanol comprising 15±1% by weight of the formulation;
water comprising 2.2±1% by weight of the formulation; and
caffeine comprising 1.3±1% by weight of the formulation.

21. The therapeutic formulation of claim 1, comprising:
hydrofluoroalkane 152a comprising 91.5±1% by weight of the formulation;
ethanol comprising 8±3.0% by weight of the formulation; and
melatonin comprising 0.449±0.5% by weight of the formulation.

22. The therapeutic formulation of claim 1, comprising:
hydrofluoroalkane 152a comprising 89±5% by weight of the formulation;
ethanol comprising 2.34±2.5% by weight of the formulation;
cannabidiol comprising 7.3±3% by weight of the formulation; and
the formulation further comprising saccharin comprising 0.06±0.05% by weight of the formulation; and
flavoring agents 0.84±0.3% by weight of the formulation.
